(19) United States
(12) Patent Application Publication
(10) Pub. No.: US 2002/0173451 A1
(43) Pub. Date: Nov. 21, 2002

(54) Method of treating cancer

(76) Inventors: Sui-Long Yao, West Windsor, NJ (US); Raymond E. Jones, Lansdale, PA (US); Deborah DeFeo-Jones, Lansdale, PA (US); David C. Heimbrook, Coopersburg, PA (US); Patricia Rhymer, Martinsville, NJ (US); Pamela J. Wasserbly, Doylestown, PA (US)

Correspondence Address: MERCK AND CO INC, P O BOX 2000, RAHWAY, NJ, 070650907

(21) Appl. No.: 09/969,244

(22) Filed: Oct. 2, 2001

Related U.S. Application Data
(60) Provisional application No. 60/242,815, filed on Oct. 24, 2000.

(51) Int. Cl. A61K 38/16; C07K 9/00; A61N 5/00

(52) U.S. Cl. 514/8; 600/1; 530/322; 530/395

(57) ABSTRACT

The present invention relates to methods of treating cancer using a combination of a PSA conjugate and radiation therapy. The methods of this invention comprise administering to a mammal in need amounts of at least one PSA conjugate, in combination with radiation therapy, either sequentially or concomitantly.

### BACKGROUND OF THE INVENTION

A neoplasm, or tumor, is an abnormal, unregulated, and disorganized proliferation of cell growth. A neoplasm is malignant, or cancerous, if it has properties of destructive growth, invasiveness and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphotics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

Cancer is now the second leading cause of death in the United States and over 8,000,000 persons in the United States have been diagnosed with cancer. In 1995, cancer accounted for 23.3% of all deaths in the United States.

Cancer is not fully understood on the molecular level. It is known that exposure of a cell to a carcinogen such as certain viruses, certain chemicals, or radiation, leads to DNA alteration that inactivates a “suppressive” gene or activates an “oncogene”. Suppressive genes are growth regulatory genes, which upon mutation, can no longer control cell growth. Oncogenes are initially normal genes (called pro-oncogenes) that by mutation or altered context of expression become transforming genes. The products of transforming genes cause inappropriate cell growth. More than twenty different normal cellular genes can become oncogenes by genetic alteration. Transformed cells differ from normal cells in many ways, including cell morphology, cell-to-cell interactions, membrane content, cytoskeletal structure, protein secretion, gene expression and mortality.

Cancer is now primarily treated with one or a combination of three types of therapies: surgery, radiation, and chemotherapy. Surgery involves the bulk removal of diseased tissue. While surgery is sometimes effective in removing tumors located at certain sites, for example, in the breast, colon and skin, it cannot be used in the treatment of tumors located in other areas, inaccessible to surgeons, nor in the treatment of disseminated neoplastic conditions such as leukemia.

Chemotherapy involves the disruption of cell replication or cell metabolism. It is used most often in the treatment of breast, lung and testicular cancer.

The adverse effects of systemic chemotherapy used in the treatment of neoplastic disease is most feared by patients undergoing treatment for cancer. Of these adverse effects nausea and vomiting are the most common and severe side effects. Other adverse side effects include cytopenia, infection, cachexia, mucositis in patients receiving high doses of chemotherapy with bone marrow rescue or radiation therapy; alopecia (hair loss); cutaneous complications such as pruritus, urticaria and angioedema; neurological complications; pulmonary and cardiac complications in patients receiving radiation or chemotherapy; and reproductive and endocrine complications (M. Abeloff, et al., Alopecia and Cutaneous Complications, in Clinical Oncology 755-56 (Abeloff, ed. 1992).

Chemotherapy-induced side effects significantly impact the quality of life of the patient and may dramatically influence patient compliance with treatment.

Additionally, adverse side effects associated with chemotherapeutic agents are generally the major dose-limiting toxicity (DLT) in the administration of these drugs. For example, mucositis is one of the major dose limiting toxicity for several anticancer agents, including the antimetabolite cytotoxic agents 5-FU, methotrexate, and antitumor antibiotics, such as doxorubicin. Many of these chemotherapy-induced side effects if severe, may lead to hospitalization, or require treatment with analgesics for the treatment of pain.

In general, radiation therapy is employed as potentially curative therapy for patients who present with clinically localized disease and are expected to live at least 10 years. For example, approximately 70% of newly-diagnosed prostate cancer patients fall into this category. Approximately 10% of these patients (7% of total patients) undergo radiation therapy. Approximately 80% of patients who have undergone radiation as their primary therapy have disease persistence or develop recurrence or metastasis within five years after treatment. Currently, most of these radiotherapy patients generally do not receive any immediate follow-up therapy. Rather, they are monitored frequently, such as for elevated Prostate Specific Antigen (“PSA”), which is the primary indicator of recurrence or metastasis in prostate cancer.

In 1999, new cases of cancer of the prostate gland were expected to be diagnosed in 179,300 men in the U.S. and 37,000 American males were expected to die from this disease (Landis, S. H. et al. CA Cancer J. Clin. 49:8-31 (1999)). Prostate cancer is the most frequently diagnosed malignancy (other than that of the skin) in U.S. men and the second leading cause of cancer-related deaths (behind lung cancer) in that group. In 2000, the Surveillance Research Program of the American Cancer Society reported its findings of the estimated cancer incidence, mortality and survival data in the United States. According to the report, the total number of cancer deaths among men had decreased for the first time in 70 years. This decrease, which occurred from 1996 to 1997 is attributed to the recent down-turns in lung and bronchus cancer deaths, prostate cancer deaths and colon and rectum cancer deaths. (R. T. Greenlee et al., CA Cancer J. Clin. (2000) 50(1):7-33).

Prostate Specific Antigen (PSA) is a single chain 33 kDa glycoprotein that is produced almost exclusively by the human prostate epithelium and occurs at levels of 0.5 to 2.0 mg/ml in human seminal fluid (Nadji, M., Taber, S. Z., Castro, A., et al. (1981) Cancer 48:1229; Papsidero, L., Kuriyama, M., Wang, M., et al. (1981), JNCI 66:37; Qui, S. D., Young, C. Y. F., Bihartz, D. L., et al. (1990), J. Urol. 144:1550; Wang, M. C., Valenzuela, L. A., Murphy, G. P., et al. (1979), Invest. Urol. 17:159; W. J. Catalona et al., New Engl. J. Med. (1991) 324(17):1156-61). The single carbohydrate unit is attached at asparagine residue number 45 and accounts for 2 to 3 kDa of the total molecular mass. PSA is a protease with chymotrypsin-like specificity (Christensson, A., Laurell, C. B., Lilja, H. (1990), Eur. J. Biochem. 194:755-763). It has been shown that PSA is mainly responsible for dissolution of the gel structure formed at ejaculation by proteolysis of the major proteins in the sperm entrapping gel, Semenogelin I and Semenogelin II, and fibronectin (Lilja, H. (1985), J. Clin. Invest. 76:1899; Lilja, H., Oldbring, J., Rannevik, G., et al. (1987), J. Clin. Invest. 80:281; McGee, R. S., Herr, J. C. (1988), Biol. Reprod. 39:499). The PSA mediated proteolysis of the gel-forming proteins generates several soluble Semenogelin I and Semenogelin II fragments and soluble fibronectin fragments with liquefaction of the ejaculate and release of progressively motile spermatoza (Lilja, H., Laurell, C. B. (1984), Scand. J. Clin. Lab. Invest. 44:447; McGee, R. S., Herr, J. C. (1987), Biol. Reprod. 37:431). Furthermore, PSA may proteolytically degrade IGFBP-3 (insulin-like growth factor binding protein 3) allowing IGF to stimulate specifically the growth of PSA secreting cells (Cohen et al., (1992) J. Clin. Endo. & Meta. 75:1046-1053).

PSA complexed to alpha 1-antichymotrypsin is the predominant molecular form of serum PSA and may account for up to 95% of the detected serum PSA (Christensson, A., Bjork, T., Nilsson, O., et al. (1993), J. Urol. 150:100-105; Lilja, H., Christensson, A., Dahlen, U. (1991), Clin. Chem. 37:1618-1625; Stenman, U. H., Leinoven, J., Alfthan, H., et al. (1991), Cancer Res. 51:222-226). The prostatic tissue (normal, benign hyperplastic, or malignant tissue) is implicated to predominantly release the mature, enzymatically active form of PSA, as this form is required for complex formation with alpha 1-antichymotrypsin (Mast, A. E., Enghild, J. J., Pizzo, S. V., et al. (1991), Biochemistry 30:1723-1730; Perlmutter, D. H., Glover, G. I., Rivetna, M., et al. (1990), Proc. Natl. Acad. Sci. USA 87:3753-3757). Therefore, in the microenvironment of prostatic PSA secreting cells the PSA is believed to be processed and secreted in its mature enzymatically active form not complexed to any inhibitory molecule. PSA also forms stable complexes with alpha 2-macroglobulin, but as this results in encapsulation of PSA and complete loss of the PSA epitopes, the in vivo significance of this complex formation is unclear. A free, noncomplexed form of PSA constitutes a minor fraction of the serum PSA (Christensson, A., Bjork, T., Nilsson, O., et al. (1993), J. Urol. 150:100-105; Lilja, H., Christensson, A., Dahlen, U. (1991), Clin. Chem. 37:1618-1625). The size of this form of serum PSA is similar to that of PSA in seminal fluid (Lilja, H., Christensson, A., Dahlen, U. (1991), Clin. Chem. 37:1618-1625) but it is yet unknown as to whether the free form of serum PSA may be a zymogen; an internally cleaved, inactive form of mature PSA; or PSA manifesting enzyme activity. However, it seems unlikely that the free form of serum PSA manifests enzyme activity, since there is considerable (100 to 1000 fold) molar excess of both unreacted alpha 1-antichymotrypsin and alpha 2-macroglobulin in serum as compared with the detected serum levels of the free 33 kDa form of PSA (Christensson, A., Bjork, T., Nilsson, O., et al. (1993), J. Urol. 150:100-105; Lilja, H., Christensson, A., Dahlen, U. (1991), Clin. Chem. 37:1618-1625).

Serum measurements of PSA are useful for monitoring the treatment of adenocarcinoma of the prostate (Duffy, M. S. (1989), Ann. Clin. Biochem. 26:379-387; Brawer, M. K. and Lange, P. H. (1989), Urol. Suppl. 5:11-16;Hara, M. and Kimura, H. (1989), J. Lab. Clin. Med. 113:541-548), although above normal serum concentrations of PSA have also been reported in benign prostatic hyperplasia and subsequent to surgical trauma of the prostate (Lilja, H., Christensson, A., Dahlen, U. (1991), Clin. Chem. 37:1618-1625). Prostate metastases are also known to secrete immunologically reactive PSA since serum PSA is detectable at high levels in prostatectomized patients showing widespread metatstatic prostate cancer (Ford, T. F., Butcher, D. N., Masters, R. W., et al. (1985), Brit. J. Urology 57:50-55). Therefore, a cytotoxic compound that could be activated by the proteolytic activity of PSA should be prostate cell specific as well as specific for PSA secreting prostate metastases.

Conjugates which comprise an oligopeptide which can be selectively cleaved by enzymatically active PSA attached, either directly or via a linker to a cytotoxic agent and which are useful in the treatment of prostate cancer and benign prostatic hyperplasia have been previously described (U.S. Pat. Nos. 5,599,686 and 5,866,679).

Current therapies for prostate cancer focus upon reducing levels of dihyfrotestosterone to decrease or prevent growth of prostate cancer. Radiation alone or in combination with surgery and/or chemotherapeutic agents is often used.

In addition to the use of digital rectal examination and transrectal ultraso9nography, prostate-specific antigen (PSA) concentration is frequently used in the diagnosis of prostate cancer.

U.S. Pat. No. 4,472,382 discloses treatment of benign prostatic hyperplasia (BPH) with an antiandrogen and certain peptides which act as LH-RH agonists. U.S. Pat. No. 4,596,797 discloses aromatase inhibitors as a method of prophylaxis and/or treatment of prostatic hyperplasia. U.S. Pat. No. 4,760,053 describes a treatment of certain cancers which combines an LHRH agonist with an antiandrogen and/or an antiestrogen and/or at least one inhibitor of sex steroid biosynthesis. U.S. Pat. No. 4,775,660 discloses a method of treating breast cancer with a combination therapy which may include surgical or chemical prevention of ovarian secretions and administering an antiandrogen and an antiestrogen. U.S. Pat. No. 4,659,695 discloses a method of treatment of prostate cancer in susceptible male animals including humans whose testicular hormonal secretions are blocked by surgical or chemical means, e.g. by use of an LHRH agonist, which comprises administering an antiandrogen, e.g. flutamide, in association with at least one inhibitor of sex steroid biosynthesis, e.g. aminoglutethimide and/or ketoconazole.

It has been previously disclosed in WO 97/38697 that oncogene protein inhibitors, such as inhibitors of farnesyl-protein inhibitors, such inhibitors of farnesyl-protein transferase or geranylgeranyl transferase, may be useful in conferring radiation sensitivity on the cell.

It is the object of the instant invention to provide a method for treating cancer, and more particularly cancer associated with cells that produce prostate specific antigen (PSA), which offers advantages over previously disclosed methods of treatment.

### SUMMARY OF THE INVENTION

A method of treating cancer, and more particularly cancer associated with cells that produce prostate specific antigen (PSA), is disclosed which is comprised of administering to a patient in need of such treatment amounts of at least one conjugate, which comprises an oligopeptide that is selectively cleaved by PSA and a cytotoxic agent, in combination with radiation therapy.

### DETAILED DESCRIPTION OF THE INVENTION

The present invention relates to a method of treating cancer, and more particularly cancer associated with cells that produce and secrete prostate specific antigen (PSA), which is comprised of administering to a patient in need of such treatment a therapeutically effective amount of at least one conjugate (hereinafter referred to as a PSA conjugate), which comprises an oligopeptide that is selectively cleaved by PSA and a cytotoxic agent, in combination with radiation therapy.

In practicing the instant method of treatment, it is understood that the PSA conjugate(s) may be administered prior to radiation therapy or they may be co-administered.

The term “treatment” refers to any process, action, application, therapy, or the like, wherein a mammal, including a human being, is subject to medical aid with the object of improving the mammal's condition, directly or indirectly.

The term “co-administration”, “co-administered” includes administration of at least one PSA conjugate either as a continuous administration over a prolonged period of time or as a single administration during the period of radiation therapy.

The phrase “therapeutically effective” is intended to qualify the amount of each agent that will achieve the goal of improvement in neoplastic disease severity and the frequency of incidence over treatment of each agent by itself, while avoiding adverse side effects typically associated with alternative therapies. A “therapeutic effect” relieves to some extent one or more of the symptoms of a neoplasia disorder. In reference to the treatment of a cancer, a therapeutic effect refers to one or more of the following: 1) reduction in the number of cancer cells; 2) reduction in tumor size; 3) inhibition (i.e. slowing to some extent, preferably stopping) of cancer cell infiltration into peripheral organs; 4) inhibition (i.e., slowing to some extent, preferably stopping) of tumor metastasis; 5) inhibition, to some extent, of tumor growth; 6) relieving or reducing to some extent on or more of the symptoms associated with the disorder; and/or 7) relieving or reducing the side effects associated with the administration of anticancer agents. “Therapeutic effective amount” is intended to qualify the amount required to achieve a therapeutic effect.

The phrase a “radiotherapeutic agent” refers to the use of electromagnetic or particulate radiation in the treatment of neoplasia. Examples of radiotherapeutic agents are provided in, but not limited to, radiation therapy and is known in the art (Hellman, Principles of Radiation Therapy, Cancer, in Principles and Practice of Oncology, 248-275 (Devita et al., ed., 4^{th} ed., vol. 1, 1993)).

The term “conjugate” or “PSA conjugate” comprises an oligopeptide, which is specifically recognized by the free prostate specific antigen (PSA) and are capable of being proteolytically cleaved by the enzymatic activity of the free prostate specific antigen, covalently bonded directly, or through a chemical linker, to a cytotoxic agent. Ideally, the cytotoxic activity of the cytotoxic agent is greatly reduced or absent when the oligopeptide containing the PSA proteolytic cleavage site is bonded directly, or through a chemical linker, to the cytotoxic agent and is intact. Also ideally, the cytotoxic activity of the cytotoxic agent increases significantly or returns to the activity of the unmodified cytotoxic agent upon proteolytic cleavage of the attached oligopeptide at the cleavage site. While it is not necessary for practicing this aspect of the invention, a preferred embodiment of this aspect of the invention is a conjugate wherein the oligopeptide, and the chemical linker if present, are detached from the cytotoxic agent by the proteolytic activity of the free PSA and any other native proteolytic enzymes present in the tissue proximity, thereby releasing unmodified cytotoxic agent into the physiological environment at the place of proteolytic cleavage. Pharmaceutically acceptable salts of the conjugates are also included.

PSA conjugates, which contain oligopeptides that are selectively cleaved by enzymatically active PSA, can be identified by a number of assays, in particularly the assays described in Examples 11-15.

In one embodiment of the instant invention, the oligopeptide component of the PSA conjugate incorporates a cyclic amino acid having a hydrophilic substituent as part of the oligopeptides, said cyclic amino acid which contributes to the aqueous solubility of the conjugate. Examples of such hydrophilic cyclic amino acids include but are not limited to hydroxylated, polyhydroxylated and alkoxylated proline and pipecolic acid moieties.

In a preferred embodiment of the invention the oligopeptide component of the PSA conjugate is characterized by having a protecting group on the terminus amino acid moiety that is not attached to the cytotoxic agent. Such protection of the terminal amino acid reduces or eliminates the enzymatic degradation of such peptidyl therapeutic agents by the action of exogenous aminopeptidases and carboxypeptidases which are present in the blood plasma of warm blooded animals. Examples of protecting groups that may be attached to the amino moiety of an N-terminus oligopeptide include, but are not limited to acetyl, benzoyl, pivaloyl, succinyl, glutaryl, hydoxyalkanoyl, polyhydroxyalkanoyl, polyethylene glycol (PEG) containing alkanoyl and the like. Examples of protecting groups that may be attached to the carboxylic acid of a C-terminus oligopeptide include, but are not limited to, formation of an organic or inorganic ester of the carboxylic acid, such as an alkyl, aralkyl, aryl, polyether ester, phosphoryl and sulfuryl, or conversion of the carboxylic acid moiety to a substituted or unsubstituted amide moiety. The N-terminus or C-terminus of the oligopeptide may also be substituted with a unnatural amino acid, such as β-alanine, or a D-amino acid, such as a D-valyl or D-alanyl group.

It is understood that the oligopeptide which is conjugated to the cytotoxic agent, whether through a direct covalent bond or through a chemical linker, does not need to be the oligopeptide that has the greatest recognition by free PSA and is most readily proteolytically cleaved by free PSA. Thus, the oligopeptide that is selected for incorporation in such conjugate will be chosen both for its selective, proteolytic cleavage by free PSA and for the cytotoxic activity of the cytotoxic agent-proteolytic residue conjugate (or, in what is felt to be an ideal situation, the unmodified cytotoxic agent) which results from such a cleavage.

Because the PSA conjugates useful in the instant compositions can be used for modifying a given biological response, cytotoxic agent component of the PSA conjugate is not to be construed as limited to classical chemical therapeutic agents. For example, the cytotoxic agent may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, b-interferon, nerve growth factor, platelet derived growth factor, tissue plasminogen activator; or, biological response modifiers such as, for example, lymphokines, interleukin-l (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

The preferred cytotoxic agents include, in general, alkylating agents, antiproliferative agents, tubulin binding agents and the like. Preferred classes of cytotoxic agents include, for example, the anthracycline family of drugs, the vinca drugs, the taxanes, the mitomycins, the bleomycins, the cytotoxic nucleosides, the pteridine family of drugs, diynenes, and the podophyllotoxins. Particularly useful members of those classes include, for example, doxorubicin, carminomycin, daunorubicin, aminopterin, methotrexate, methopterin, dichloro-methotrexate, mitomycin C, porfiromycin, paclitaxel, docetaxel, 5-fluorouracil, 6-mercaptopurine, cytosine arabinoside, podophyllotoxin, or podophyllotoxin derivatives such as etoposide or etoposide phosphate, melphalan, vinblastine, vincristine, leurosidine, vindesine, leurosine and the like. Other useful cytotoxic agents include estramustine, cisplatin and cyclophosphamide. One skilled in the art may make chemical modifications to the desired cytotoxic agent in order to make reactions of that compound more convenient for purposes of preparing PSA conjugates of the invention.

Preferably the cytotoxic agent component of the PSA conjugate is selected from a member of a class of cytotoxic agents selected from the vinca alkaloid drugs and the anthracyclines.

PSA conjugates that are useful in the methods of the instant invention and are identified by the properties described hereinabove include:

a) a compound represented by the formula I:

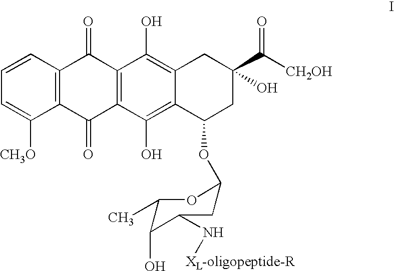

wherein:

oligopeptide is an oligopeptide which is specifically recognized by the free prostate specific antigen (PSA) and is capable of being proteolytically cleaved by the enzymatic activity of the free prostate specific antigen;

X_{L} is absent or is an amino acid selected from:

a) phenylalanine,

b) leucine,

c) valine,

d) isoleucine,

e) (2-naphthyl)alanine,

f) cyclohexylalanine,

g) diphenylalanine,

h) norvaline, and

j) norleucine;

R is hydrogen or —(C═O)R¹; and

R¹ is C₁-C₆-alkyl or aryl,

or the pharmaceutically acceptable salt thereof;

b) a compound represented by the formula II:

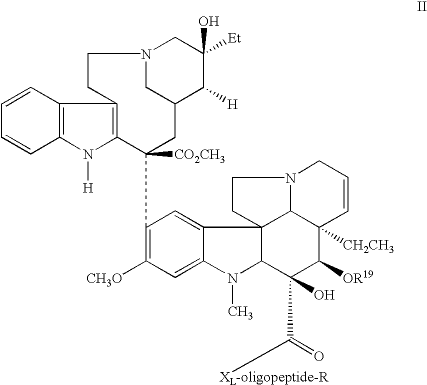

wherein:

oligopeptide is an oligopeptide which is specifically recognized by the free prostate specific antigen (PSA) and is capable of being proteolytically cleaved by the enzymatic activity of the free prostate specific antigen;

X_{L} is absent or is an amino acid selected from:

a) phenylalanine,

b) leucine,

c) valine,

d) isoleucine,

e) (2-naphthyl)alanine,

f) cyclohexylalanine,

g) diphenylalanine,

h) norvaline, and

j) norleucine; or

X_{L} is —NH—(CH₂)ₙ—NH—;

R is hydrogen or —(C═O)R¹;

R¹ is C₁-C₆-alkyl or aryl;

R¹⁹ is hydrogen or acetyl; and

n is 1, 2, 3, 4 or 5,

or the pharmaceutically acceptable salt thereof;

c) a compound represented by the formula III:

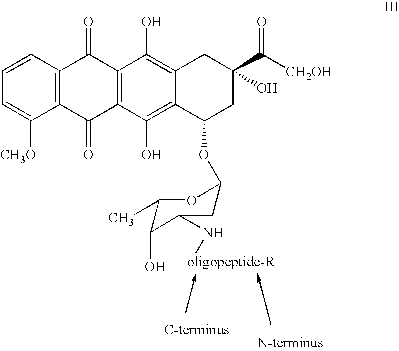

wherein:

oligopeptide is an oligopeptide which is selectively recognized by the free prostate specific antigen (PSA) and is capable of being proteolytically cleaved by the enzymatic activity of the free prostate specific antigen, wherein the oligopeptide comprises a cyclic amino acid of the formula:

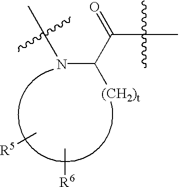

and wherein

the C-terminus carbonyl is covalently bound to the amine of doxorubicin;

R is selected from

a) hydrogen,

b) —(C═O)R^{1a},

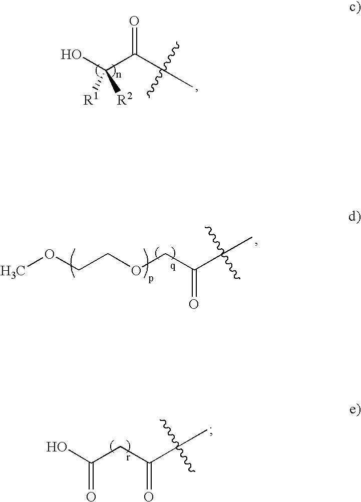

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated aryl, polyhydroxylated aryl or aryl;

R⁵ is selected from HO— and C₁-C₆ alkoxy;

R⁶ is selected from hydrogen, halogen, C₁-C₆ alkyl, HO— and C₁-C₆ alkoxy; and

n is 1,2,3 or 4;

p is zero or an integer between 1 and 100;

q is 0 or 1, provided that if p is zero, q is 1;

r is an integer between 1 and 10; and

t is 3 or 4;

or a pharmaceutically acceptable salt thereof;

d) a compound represented by the formula IV:

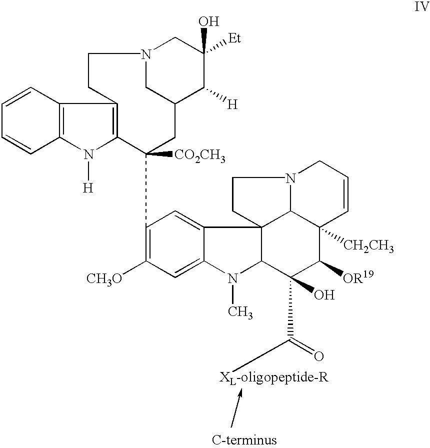

wherein:

oligopeptide is an oligopeptide which is specifically recognized by the free prostate specific antigen (PSA) and is capable of being proteolytically cleaved by the enzymatic activity of the free prostate specific antigen, and the oligopeptide comprises a cyclic amino acid of the formula:

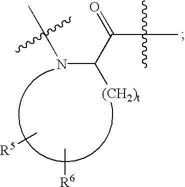

X_{L} is —NH—(CH₂)ᵤ—NH—;

R is selected from

a) hydrogen,

b) —(C═O)R^{1a},

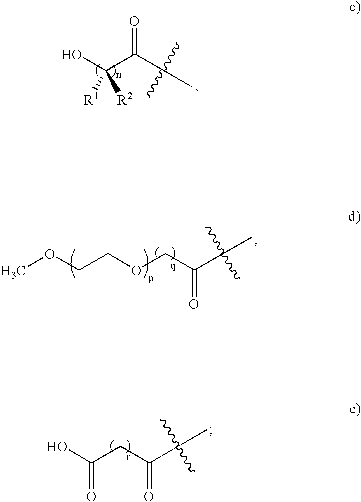

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated aryl, polyhydroxylated aryl or aryl;

R⁵ is selected from HO— and C₁-C₆ alkoxy;

R⁶ is selected from hydrogen, halogen, C₁-C₆ alkyl, HO— and C₁-C₆ alkoxy; and

R¹⁹ is hydrogen, (C 1-C₃ alkyl)—CO, or chlorosubstituted (C 1-C₃ alkyl)—CO;

n is 1,2,3 or 4;

p is zero or an integer between 1 and 100;

q is 0 or 1, provided that if p is zero, q is 1;

r is 1,2 or 3;

t is 3 or 4;

u is 1,2,3,4 or 5;

or the pharmaceutically acceptable salt thereof;

e) a compound represented by the formula V:

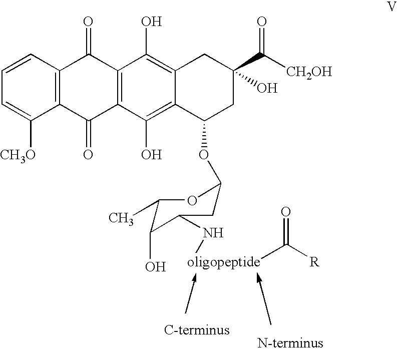

wherein:

oligopeptide is an oligopeptide which is selectively recognized by the free prostate specific antigen (PSA) and is capable of being proteolytically cleaved by the enzymatic activity of the free prostate specific antigen, and wherein the C-terminus carbonyl is covalently bound to the amine of doxorubicin and the N-terminus amine is covalently bound to the carbonyl of the blocking group;

R is selected from

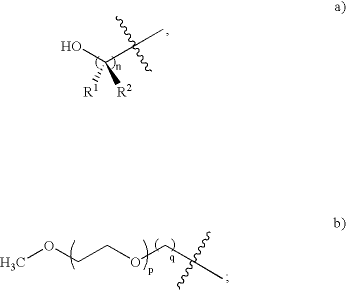

R¹ and R² are independently selected from: hydrogen, OH, C₁-C₆ alkyl, C₁-C₆ alkoxy, C₁-C₆ aralkyl and aryl;

n is 1, 2, 3 or 4;

p is zero or an integer between 1 and 100;

q is 0 or 1, provided that if p is zero, q is 1;

or the pharmaceutically acceptable salt thereof;

f) a compound represented by the formula VI:

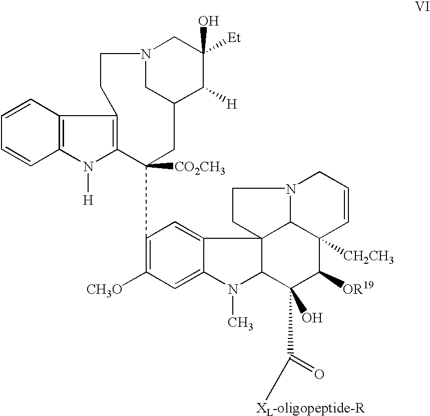

wherein:

oligopeptide is an oligopeptide which is specifically recognized by the free prostate specific antigen (PSA) and is capable of being proteolytically cleaved by the enzymatic activity of the free prostate specific antigen;

X_{L} is —NH—(CH₂)ᵣ—NH—;

R is selected from

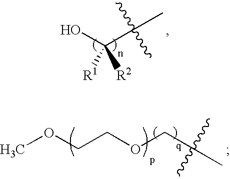

R¹ and R² are independently selected from: hydrogen, OH, C₁-C₆ alkyl, C₁-C₆ alkoxy, C₁-C₆ aralkyl and aryl;

R¹⁹ is hydrogen, (C₁-C₃ alkyl)—CO, or chlorosubstituted (C₁-C₃ alkyl)—CO;

n is 1,2,3 or 4;

p is zero or an integer between 1 and 100;

q is 0 or 1, provided that if p is zero, q is 1;

r is 1,2,3,4 or 5;

or the pharmaceutically acceptable salt thereof;

g) a compound represented by the formula VII:

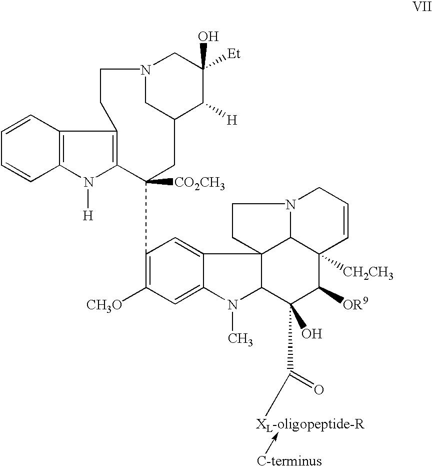

wherein:

oligopeptide is an oligopeptide which is specifically recognized by the free prostate specific antigen (PSA) and is capable of being proteolytically cleaved by the enzymatic activity of the free prostate specific antigen,

X_{L} is —NH—(CH₂)ᵤ—W—(CH₂)ᵤ—NH—;

R is selected from

a) hydrogen,

b) —(C═O)R^{1a},

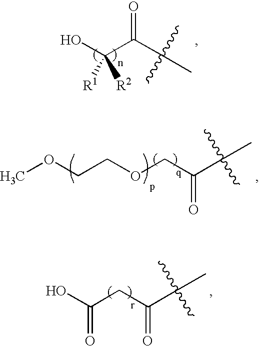

f) ethoxysquarate, and

g) cotininyl;

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;

W is selected from cyclopentyl, cyclohexyl, cycloheptyl or bicyclo[2.2.2]octanyl;

n is 1,2,3 or 4;

p is zero or an integer between 1 and 100;

q is 0 or 1, provided that if p is zero, q is 1;

r is 1, 2 or 3;

t is 3 or 4;

u is 0, 1, 2 or 3;

or the pharmaceutically acceptable salt thereof; and

h) a compound represented by the formula VIII:

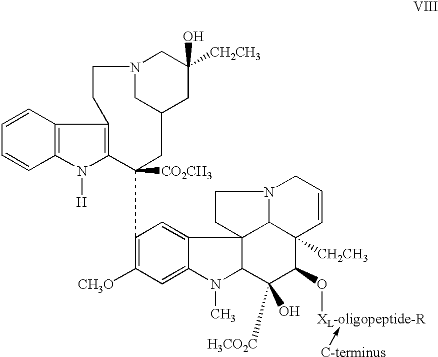

wherein:

oligopeptide is an oligopeptide which is specifically recognized by the free prostate specific antigen (PSA) and is capable of being proteolytically cleaved by the enzymatic activity of the free prostate specific antigen,

X_{L} is selected from: a bond, —C(O)—(CH₂)ᵤ—W—(CH₂)ᵤ—O— and —C(O)—(CH₂)ᵤ—W—(CH₂)ᵤ—NH—;

R is selected from

a) hydrogen,

b) —(C═O)R^{1a},

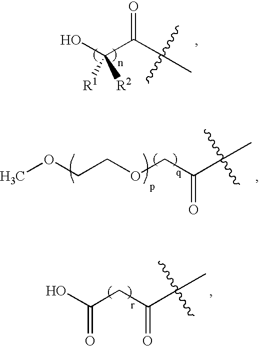

f) ethoxysquarate, and

g) cotininyl;

R¹ and R² are independently selected from: hydrogen, OH, C₁-C₆ alkyl, C₁-C₆ alkoxy, C₁-C₆ aralkyl and aryl;

R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;

W is selected from a branched or straight chain C₁ -C₆-alkyl, cyclopentyl, cyclohexyl, cycloheptyl or bicyclo[2.2.2]octanyl;

n is 1,2,3 or 4;

p is zero or an integer between 1 and 100;

q is 0 or 1, provided that if p is zero, q is 1;

r is 1,2 or 3;

t is 3 or 4;

u is 0, 1, 2 or 3;

or the pharmaceutically acceptable salt or optical isomer thereof.

Examples of compounds which are PSA conjugates include the following:

i)

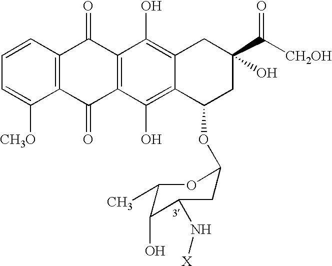

wherein X is:

| | |
|---|---|
| AsnLysIleSerTyrGlnSer- | (SEQ.ID.NO.:1), |
| AsnLysIleSerTyrGlnSerSer- | (SEQ.ID.NO.:2), |
| AsnLysIleSerTyrGlnSerSerSer- | (SEQ.ID.NO.:3), |
| AsnLysIleSerTyrGlnSerSerSerThr- | (SEQ.ID.NO.:4), |
| AsnLysIleSerTyrGlnSerSerSerThrGlu- | (SEQ.ID.NO.:5), |
| AlaAsnLysIleSerTyrGlnSerSerSerThrGlu- | (SEQ.ID.NO.:6), |
| Ac-AlaAsnLysIleSerTyrGlnSerSerSerThr- | (SEQ.ID.NO.:7), |
| Ac-AlaAsnLysIleSerTyrGlnSerSerSerThrLeu- | (SEQ.ID.NO.:8), |
| Ac-AlaAsnLysAlaSerTyrGlnSerAlaSerThrLeu- | (SEQ.ID.NO.:9), |
| Ac-AlaAsnLysAlaSerTyrGlnSerAlaSerLeu- | (SEQ.ID.NO.:10), |
| Ac-AlaAsnLysALaSerTyrGlnSerSerSerLeu- | (SEQ.ID.NO.:11), |
| Ac-AlaAsnLysAlaSerTyrGlnSerSerLeu- | (SEQ.ID.NO.:12), |
| Ac-SerTyrGlnSerSerSerLeu- | (SEQ.ID.NO.:13), |
| Ac-hArgTyrGlnSerSerSerLeu- | (SEQ.ID.NO.:14). |
| Ac-LysTyrGlnSerSerSerLeu- | (SEQ.ID.NO.:15), or |
| Ac-LysTyrGlnSerSerNle- | (SEQ.ID.NO.:16); |

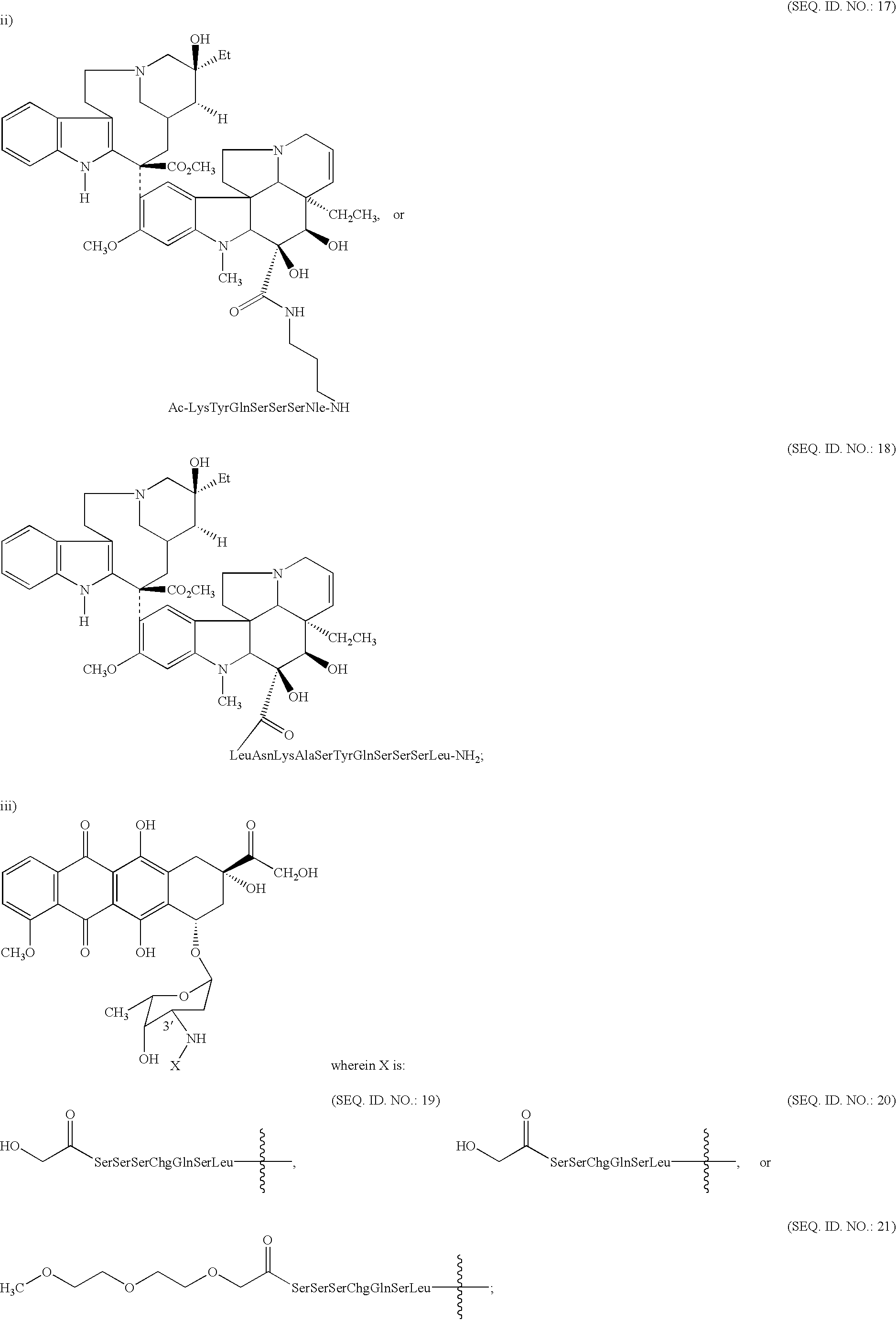

Preferably the method of the instant invention comprises the PSA conjugate

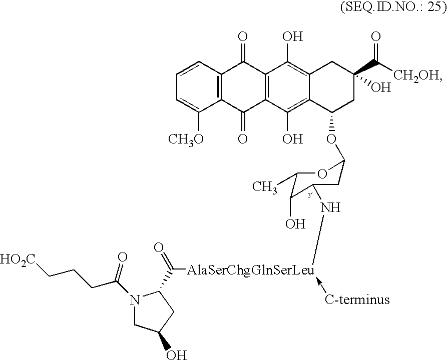

or the pharmaceutically acceptable salt thereof.

Compounds which are PSA conjugates and are therefore useful in the present invention, and methods of synthesis thereof, can be found in the following patents, pending applications and publications, which are herein incorporated by reference:

U.S. Pat. No. 5,599,686 granted on Feb. 4, 1997;

WO 96/00503 (Jan. 11, 1996); U.S. Ser. No. 08/404,833 filed on Mar. 15, 1995; U.S. Ser. No. 08/468,161 filed on Jun. 6, 1995;

U.S. Pat. No. 5,866,679 granted on Feb. 2, 1999;

WO 98/10651 (Mar. 19, 1998); U.S. Ser. No. 08/926,412 filed on Sep. 9, 1997;

WO 98/18493 (May 7, 1998); U.S. Pat. No. 5,948,750, granted on Sep. 7, 1999

U.S. Ser. No. 09/112,656 filed on Jul. 9, 1998; U.S. Ser. No. 60/052,195 filed on Jul. 10, 1997; and

U.S. Ser. No. 09/193,365 filed on Nov. 17, 1998; U.S. Ser. No. 60/067,110 filed on Dec. 2, 1997.

Compounds which are described as prodrugs wherein the active therapeutic agent is release by the action of enzymatically active PSA and therefore may be useful in the present invention, and methods of synthesis thereof, can be found in the following patents, pending applications and publications, which are herein incorporated by reference: WO 98/52966 (Nov. 26, 1998).

All patents, publications and pending patent applications identified are hereby incorporated by reference.

With respect to the compounds of formulas I through VIII the following definitions apply:

“Alkyl” means linear branched and cyclic structures, and combinations thereof, containing the indicated number of carbon atoms. Examples of alkyl groups include methyl, ethyl, propyl, isopropyl, butyl, s- and t-butyl, pentyl, hexyl, heptyl, octyl, nonyl, undecyl, dodecyl, tridecyl, tetradecyl, pentadecyl, eicosyl, 3,7-diethyl-2,2-dimethyl-4-propylnonyl, cyclopropyl, cyclopentyl, cycloheptyl, adamantyl, cyclododecylmethyl, 2-ethyl-1-bicyclo[4.4.0]decyl and the like.

As used herein, “alkyl” and the alkyl portion of aralkyl and similar terms, is intended to include both branched and straight-chain saturated aliphatic hydrocarbon groups having the specified number of carbon atoms; “alkoxy” represents an alkyl group of indicated number of carbon atoms attached through an oxygen bridge.

As used herein, “cycloalkyl” is intended to include non-aromatic cyclic hydrocarbon groups having the specified number of carbon atoms. Examples of cycloalkyl groups include cyclopropyl, cyclobutyl, cyclopentyl, cyclohexyl and the like.

“Halogen” or “halo” as used herein means fluoro, chloro, bromo and iodo.

“Fluoro alkyl” means alkyl groups in which one or more hydrogen is replaced by fluorine. Examples are —CF₃, —CH₂CH₂F, —CH₂CF₃, c-Pr-F₅, c-Hex-F₁₁ and the like. Similarly, fluoroalkoxy means linear, branched and cyclic structures, with the indicated number of carbon atoms.

For purposes of this specification “Alkoxy” means alkoxy groups of the indicated number of carbon atoms of a straight, branched, or cyclic configuration. Examples of alkoxy groups include methoxy, ethoxy, propoxy, isopropoxy, cyclopropyloxy, cyclohexyloxy, and the like.

“Alkylthio” means alkylthio groups of the indicated number of carbon atoms of a straight, branched or cyclic configuration. Examples of alkylthio groups include methylthio, propylthio, isopropylthio, cycloheptylthio, etc. By way of illustration, the propylthio group signifies —SCH₂CH₂CH₃.

As used herein, “aryl,” and the aryl portion of aralkyl and aroyl, is intended to mean any stable monocyclic or bicyclic carbon ring of up to 7 members in each ring, wherein at least one ring is aromatic. Examples of such aryl elements include phenyl, naphthyl, tetrahydro-naphthyl, indanyl, biphenyl, phenanthryl, anthryl or acenaphthyl.

Unless otherwise defined, heteroaryl includes aromatic and partially aromatic groups which contain one or more heteroatoms. Examples of this type are thiophene, purine, imidazopyridine, pyridine, oxazole, thiazole, oxazine, pyrazole, tetrazole, imidazole, pyridine, pyrimidine, pyrazine and triazine. Examples of partially aromatic groups are tetrahydro-imidazo[4,5-c]pyridine, phthalidyl and saccharinyl, as defined below.

In situations in which a term occurs two or more times, the definition of the term in each occurrence is independent of the definition in each additional occurrence.

With respect to the compounds of formulas IV through XI the following definitions apply:

As used herein, “oligopeptide” is preferably a peptide comprising from about 5 amino acids to about 100 amino acids. More preferably, “oligopeptide” is a peptide comprising from about 5 amino acids to about 15 amino acids.

The terms “selective” and “selectively” as used in connection with recognition by PSA and the proteolytic PSA cleavage mean a greater rate of cleavage of an oligopeptide component of the instant invention by free PSA relative to cleavage of an oligopeptide which comprises a random sequence of amino acids. Therefore, the oligopeptide component of the instant invention is a preferred substrate of free PSA. The terms “selective” and “selectively” also indicate that the oligopeptide is proteolytically cleaved by free PSA between two specific amino acids in the oligopeptide.

As used herein, the term “hydroxylated” represents substitution on a substitutable carbon of the ring system being so described by a hydroxyl moiety. As used herein, the term “poly-hydroxylated” represents substitution on two or more substitutable carbon of the ring system being so described by 2, 3 or 4 hydroxyl moieties.

As used herein, the term “chlorosubstituted C₁-C₃-alkyl-CO-” represents a acyl moiety having the designated number of carbon atoms attached to a carbonyl moiety wherein one of the carbon atoms is substituted with a chlorine. Example of such chlorosubstituted elements include but are not limited to chloroacetyl, 2-chloropropionyl, 3-chloropropionyl and 2-chlorobutyroyl.

As used herein, the term “PEG” represents certain polyethylene glycol containing substituents having the designated number of ethyleneoxy subunits. Thus the term PEG(2) represents

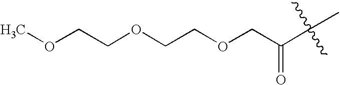

and the term PEG(6) represents

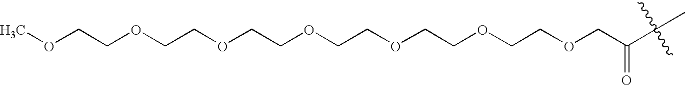

As used herein, the term “(d)(2,3-dihydroxypropionyl)” represents the following structure:

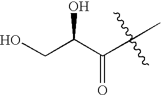

As used herein, the term “(2R,3S) 2,3,4-trihydroxybutanoyl” represents the following structure:

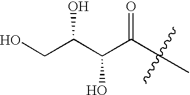

As used herein, the term “quinyl” represents the following structure:

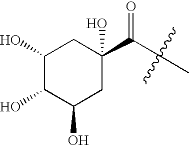

or the diastereomer thereof.

As used herein, the term “cotininyl” represents the following structure:

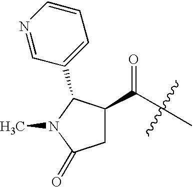

or the diastereomer thereof.

As used herein, the term “gallyl” represents the following structure:

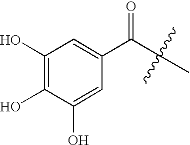

As used herein, the term “4-ethoxysquarate” represents the following structure:

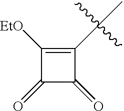

The structure

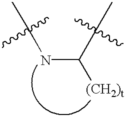

represents a cyclic amine moiety having 5 or 6 members in the ring, such a cyclic amine which may be optionally fused to a phenyl or cyclohexyl ring. Examples of such a cyclic amine moiety include, but are not limited to, the following specific structures:

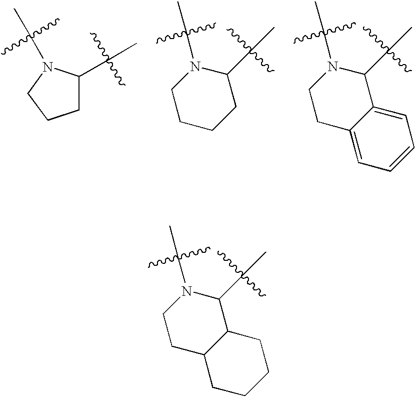

The pharmaceutically acceptable salts of the compounds of this invention include the conventional non-toxic salts of the compounds of this invention as formed, e.g., from non-toxic inorganic or organic acids. For example, such conventional non-toxic salts include those derived from inorganic acids such as hydrochloric, hydrobromic, sulfuric, sulfamic, phosphoric, nitric and the like: and the salts prepared from organic acids such as acetic, propionic, succinic, glycolic, stearic, lactic, malic, tartaric, citric, ascorbic, pamoic, maleic, hydroxymaleic, phenyl-acetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

It is intended that the definition of any substituent or variable (e.g., R¹⁰, Z, n, etc.) at a particular location in a molecule be independent of its definitions elsewhere in that molecule. Thus, —N(R¹⁰)₂ represents —NHH, —NHCH₃, —NHC₂H₅, etc. It is understood that substituents and substitution patterns on the compounds of the instant invention can be selected by one of ordinary skill in the art to provide compounds that are chemically stable and that can be readily synthesized by techniques known in the art as well as those methods set forth below.

The compounds are useful in various pharmaceutically acceptable salt forms. The term “pharmaceutically acceptable salt” refers to those salt forms which would be apparent to the pharmaceutical chemist. i.e., those which are substantially non-toxic and which provide the desired pharmacokinetic properties, palatability, absorption, distribution, metabolism or excretion. Other factors, more practical in nature, which are also important in the selection, are cost of the raw materials, ease of crystallization, yield, stability, hygroscopicity and flowability of the resulting bulk drug. Conveniently, pharmaceutical compositions may be prepared from the active ingredients in combination with pharmaceutically acceptable carriers.

The pharmaceutically acceptable salts of the compounds of this invention include the conventional non-toxic salts of the compounds of this invention as formed, e.g., from non-toxic inorganic or organic acids. For example, such conventional non-toxic salts include those derived from inorganic acids such as hydrochloric, hydrobromic, sulfuric, sulfamic, phosphoric, nitric and the like: and the salts prepared from organic acids such as acetic, propionic, succinic, glycolic, stearic, lactic, malic, tartaric, citric, ascorbic, pamoic, maleic, hydroxymaleic, phenyl-acetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

The term “pharmaceutically acceptable salts” also refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like, and basic ion exchange resins.

Suitable pharmaceutically acceptable salts of the compounds of use in the present invention include acid addition salts which may, for example, be formed by mixing a solution of the compound with a solution of a pharmaceutically acceptable non-toxic acid such as hydrochloric acid, fumaric acid, maleic acid, succinic acid, acetic acid, citric acid, tartaric acid, carbonic acid, phosphoric acid or sulphuric acid. Salts of amine groups may also comprise the quaternary ammonium salts in which the amino nitrogen atom carries an alkyl, alkenyl, alkynyl or aralkyl group. Where the compound carries an acidic group, for example a carboxylic acid group, the present invention also contemplates salts thereof, preferably non-toxic pharmaceutically acceptable salts thereof, such as the sodium, potassium and calcium salts thereof.

The pharmaceutically acceptable salts of the compounds of this invention can be synthesized from the compounds of this invention which contain a basic moiety by conventional chemical methods. Generally, the salts are prepared by reacting the free base with stoichiometric amounts or with an excess of the desired salt-forming inorganic or organic acid in a suitable solvent or solvent combination, or various combinations of solvents available Nₐ-Z-L-2,3-diaminopropionic acid (Fluka) as a starting material is preferred.

It is intended that the definition of any substituent or variable (e.g., R¹⁰, Z, n, etc.) at a particular location in a molecule be independent of its definitions elsewhere in that molecule. Thus, —N(R¹⁰)₂ represents —NHH, —NHCH₃, —NHC₂H₅, etc. It is understood that substituents and substitution patterns on the compounds of the instant invention can be selected by one of ordinary skill in the art to provide compounds that are chemically stable and that can be readily synthesized by techniques known in the art as well as those methods set forth below.

Abbreviations used in the description of the chemistry and in the Examples that follow are:

| | |
|---|---|
| Ac₂O | Acetic anhydride; |
| Boc | t-Butoxycarbonyl; |
| Bzl | Benzyl; |
| DABCO | 1,4-Diazabicyclo[2.2.2]octane; |
| or Dabco | |
| DBU | 1,8-diazabicyclo[5.4.0]undec-7-ene; |
| DCC | 1,3-Dicyclohexylcarbodiimide; |
| DIEA | Diisopropylethylamine; |
| DMAP | 4-Dimethylaminopyridine; |
| DME | 1,2-Dimethoxyethane; |
| DMF | Dimethylformamide; |
| DMSO | Dimethylsulfoxide; |
| DPPA | Diphenyiphosphoryl azide; |
| EDC | 1-(3-dimethylaminopropyl)-3-ethyl-carbodiimide- |
| | hydrochloride; |
| HOAC | Acetic acid; |
| HOBT | 1-Hydroxybenzotriazole hydrate; |
| Et₃N | Triethylamine; |
| EtOAc | Ethyl acetate; |
| FAB | Fast atom bombardment; |
| HOOBT | 3-Hydroxy-1,2,2-benzotriazin-4(3H)-one; |
| HPLC | High-performance liquid chromatography; |
| MCPBA | m-Chloroperoxybenzoic acid; |
| MIIBK | Methyl isobutyl ketone; |
| MsCl | Methanesulfonyl chloride; |
| MTBE | Methyl-t-butyl ether; |
| NaHMDS | Sodium bis(trimethylsilyl)amide; |
| NMP | 1-methyl-2-pyrrolidinone; |
| Py | Pyridine; |
| TEA | Triethanolamine; |
| TFA | Trifluoroacetic acid; |
| THF | Tetrahydrofuran; |
| TLC | Thin Layer Chromatography. |

The PSA conjugates of formulae I, III and V can be synthesized in accordance with Schemes 1-5, in addition to other standard manipulations such as ester hydrolysis, cleavage of protecting groups, etc., as may be known in the literature or exemplified in the experimental procedures.

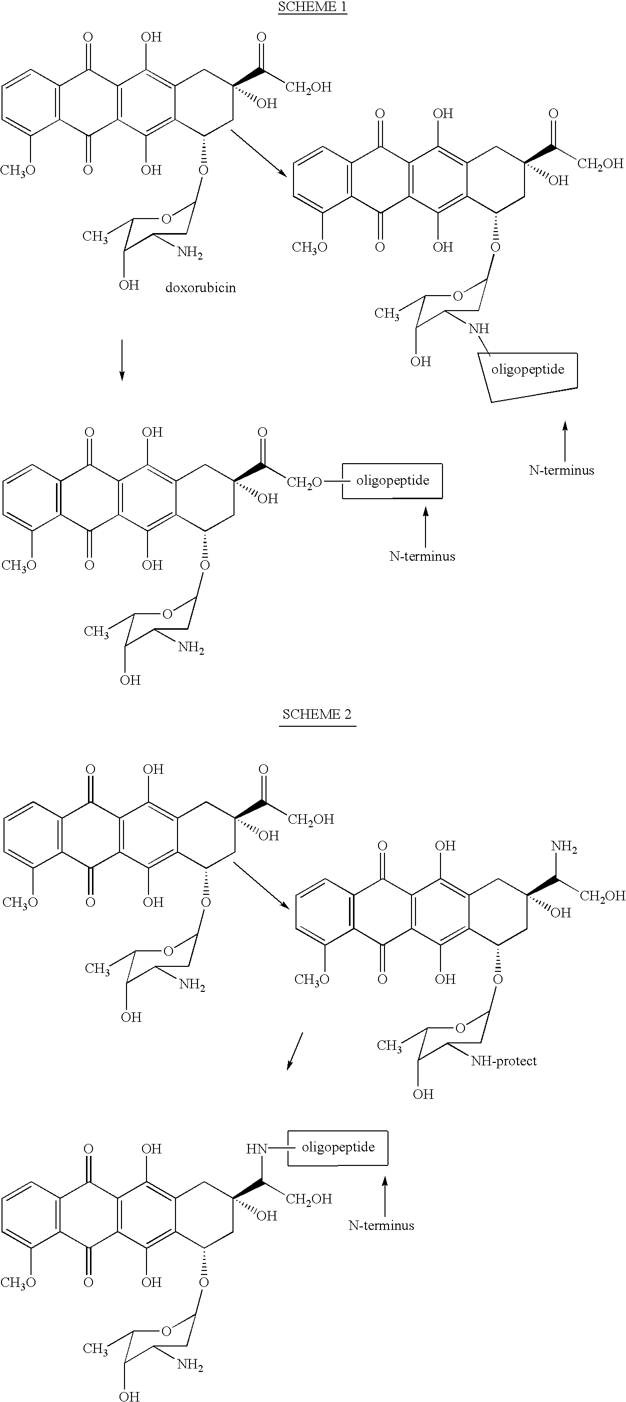

Scheme 6 illustrates preparation of conjugates utilized in the instant method of treatment wherein the oligopeptides are combined with the vinca alkaloid cytotoxic agent vinblastine. Attachment of the N-terminus of the oligopeptide to vinblastine is illustrated (S. P. Kandukuri et al. J. Med. Chem. 28:1079-1088 (1985)).

Scheme 7 illustrates preparation of conjugates of the oligopeptides of the instant invention and the vinca alkaloid cytotoxic agent vinblastine wherein the attachment of vinblastine is at the C-terminus of the oligopeptide. The use of the 1,3-diaminopropane linker is illustrative only; other spacer units between the carbonyl of vinblastine and the C-terminus of the oligopeptide are also envisioned. Furthermore, Scheme 7 illustrates a synthesis of conjugates wherein the C-4-position hydroxy moiety is reacetylated following the addition of the linker unit. Applicants have discovered that a desacetyl vinblastine conjugate is also useful in the instant methods. This conjugate may be prepared by eliminating the steps shown in Scheme 7 of protecting the primary amine of the linker and reacting the intermediate with acetic anhydride, followed by deprotection of the amine. Conjugation of the oligopeptide at other positions and functional groups of vinblastine may be readily accomplished by one of ordinary skill in the art and is also expected to provide compounds useful in the instant methods of treatment.

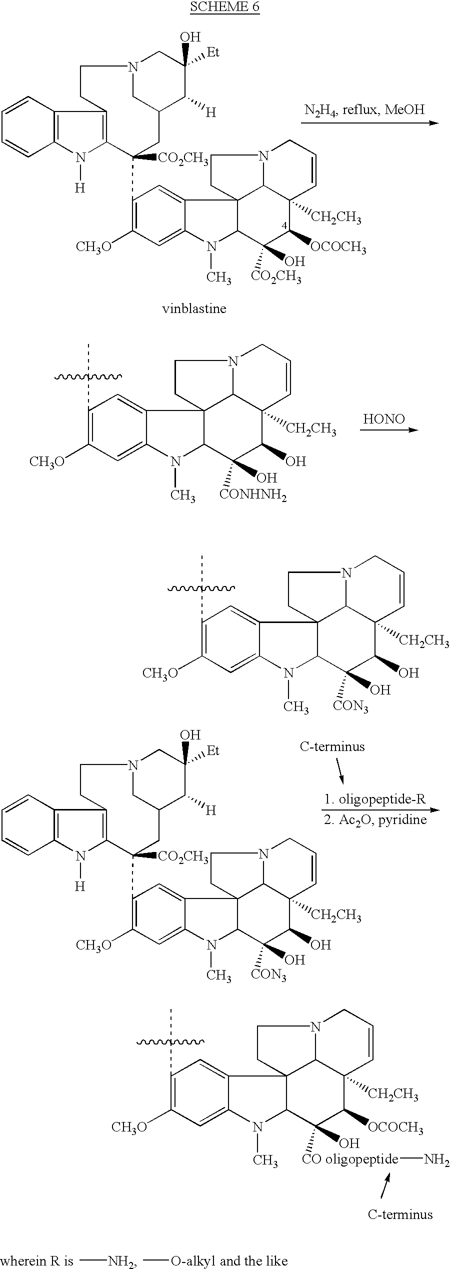

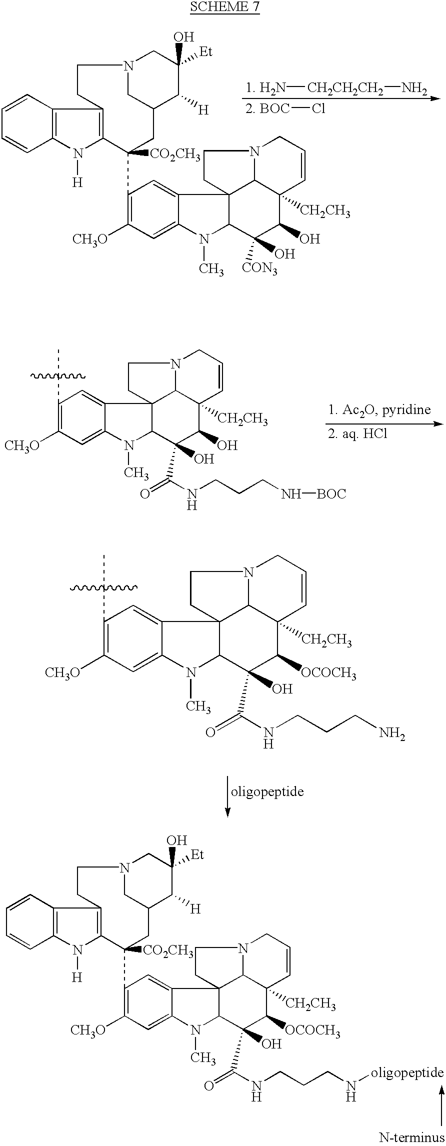

The PSA conjugates of formula III and V can be synthesized in accordance with Schemes 8-12, in addition to other standard manipulations such as ester hydrolysis, cleavage of protecting groups, etc., as may be known in the literature or exemplified in the experimental procedures.

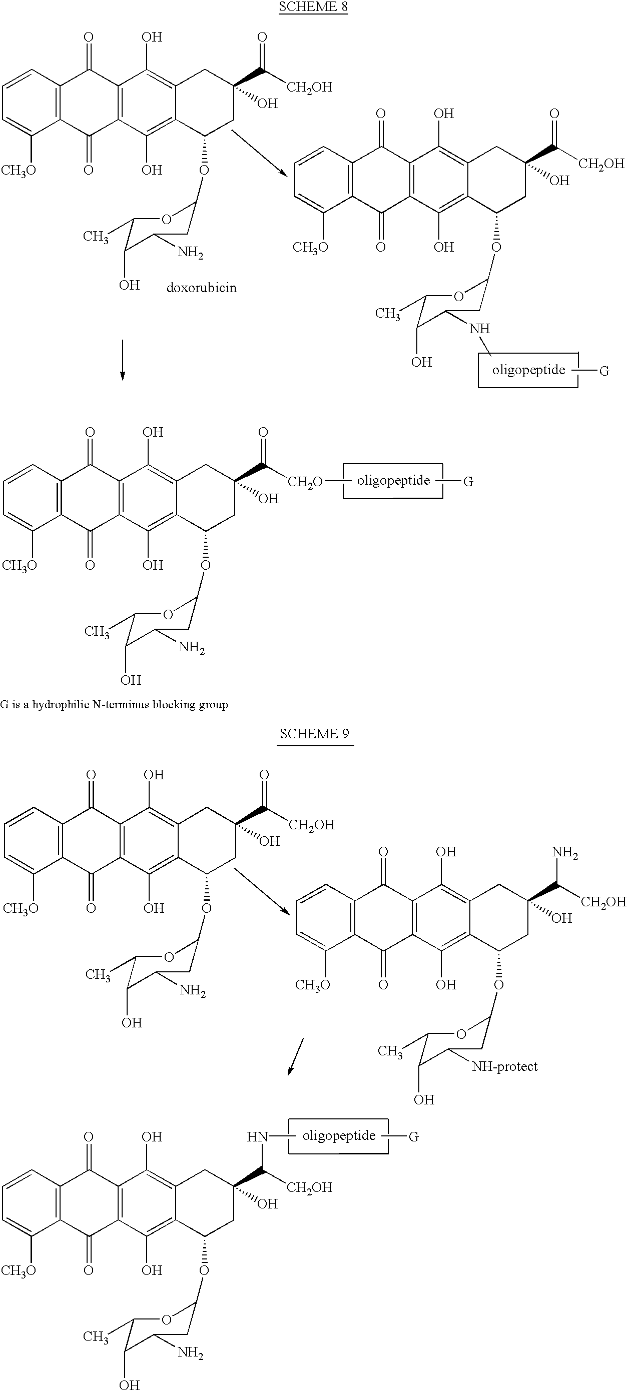

Scheme 13 illustrates preparation of PSA conjugates of the formula VI wherein the attachment of vinblastine is at the C-terminus of the oligopeptide. Furthermore, Scheme 13 illustrates a synthesis of conjugates wherein the C-4-position hydroxy moiety is reacetylated following the addition of the linker unit. Applicants have discovered that the desacetyl vinblastine conjugate is also efficacious and may be prepared by eliminating the steps shown in Scheme 13of protecting the primary amine of the linker and reacting the intermediate with acetic anhydride, followed by deprotection of the amine. Conjugation of the oligopeptide at other positions and functional groups of vinblastine may be readily accomplished by one of ordinary skill in the art and is also expected to provide compounds useful in the treatment of prostate cancer.

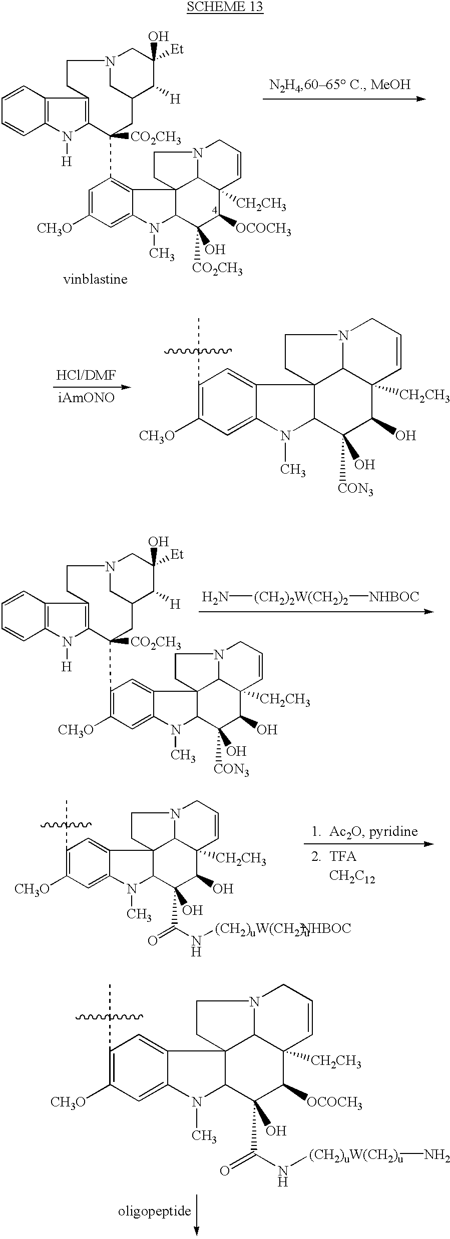

The PSA conjugates of formula VII can be synthesized in accordance with Schemes 14-15, in addition to other standard manipulations such as ester hydrolysis, cleavage of protecting groups, etc., as may be known in the literature or exemplified in the experimental procedures.

Reaction Scheme 14 illustrates preparation of conjugates of the oligopeptides of the instant invention and the vinca alkaloid cytotoxic agent vinblastine wherein the attachment of the oxygen of the 4-desacetylvinblastine is at the C-terminus of the oligopeptide. While other sequences of reactions may be useful in forming such conjugates, it has been found that initial attachment of a single amino acid to the 4-oxygen and subsequent attachment of the remaining oligopeptide sequence to that amino acid is a preferred method. It has also been found that 3,4-dihydro-3-hydroxy-4-oxo-1,2,3-benzotriazine (ODHBT) may be utilized in place of HOAt in the final coupling step.

Reaction Scheme 15 illustrates preparation of conjugates of the oligopeptides of the instant invention wherein a hydroxy alkanolyl acid is used as a linker between the vinca drug and the oligopeptide.

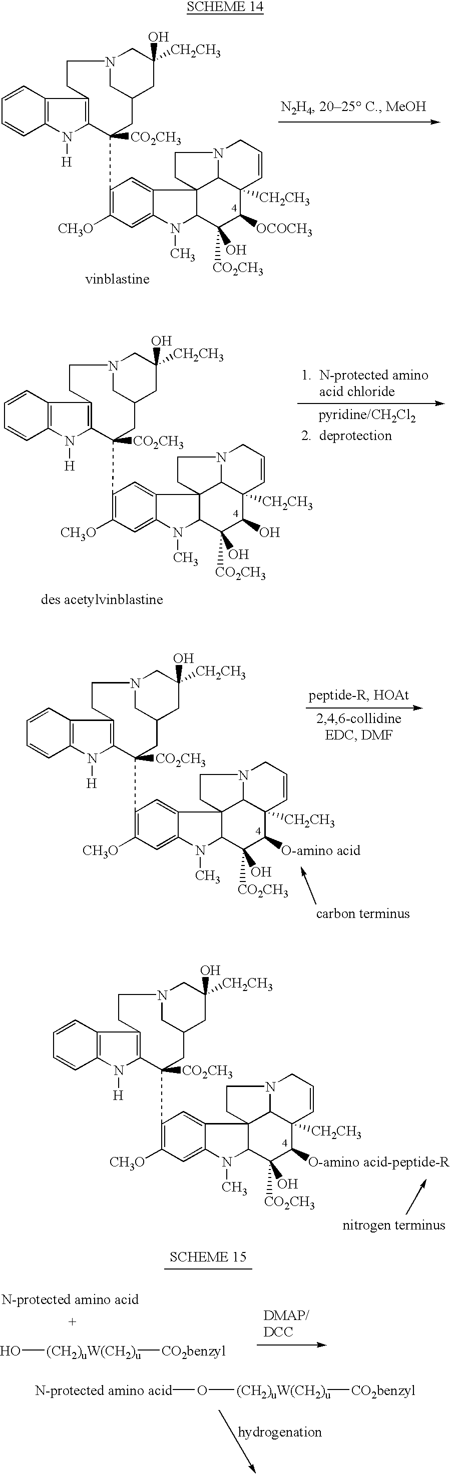

A benefit of the present invention is lowering the dose of the radiation therapies administered to a mammal to decrease the incidence of adverse effects associated with higher dosages.

By lowering the incidence of adverse effects, an improvement in the quality of life of a patient undergoing treatment for cancer is achieved. Further benefits of lowering the incidence of adverse effects include an improvement in patient compliance, and a reduction in the number of hospitalizations needed for the treatment of adverse effects.

Alternatively, the methods and combination of the present invention can also maximize the therapeutic effect at higher doses.

The phrase “combination therapy” (or “co-therapy”) embraces administration of each agent or therapy in a sequential manner in a regimen that will provide beneficial effects of the combination, and co-administration of these agents or therapies in a substantially simultaneous manner, such as in a single capsule having a fixed ratio of these active agents or in multiple, separate capsules for each agent. Combination therapy also includes combinations where the individual elements may be administered at different times and/or by different routes but which act in combination to provide a beneficial effect by co-action or pharmacokinetic and pharmacodynamic effect of each agent or tumor treatment approaches of the combination therapy.

Generally, radiation therapy has been combined temporally with chemotherapy to improve the outcome of treatment. There are various terms to describe the temporal relationship of administering radiation therapy and chemotherapy. The following examples are the preferred treatment regimens and are generally known by those skilled in the art and are provided for illustration only and are not intended to limit the use of other combination. “Sequential” radiation therapy and chemotherapy refers to the administration of chemotherapy and radiation therapy separately in time in order to allow the separate administration of either chemotherapy or radiation therapy. “Concomitant” radiation therapy and chemotherapy refers to the administration of chemotherapy and radiation therapy on the same day. Finally, “alternating” radiation therapy and chemotherapy refers to the administration of radiation therapy on the days in which chemotherapy would not have been administered if it was given alone.

Radiation therapy is based on the principle that high-dose radiation delivered to a target area will result in the death of reproductive cells in both tumor and normal tissues. The radiation dosage regiment is generally defined in terms of radiation absorbed dose (rad), time and fractionation, and must be carefully defined by the oncologist. The amount of radiation a patient receives will depend on various consideration, but the two most important considerations are the location of the tumor in relation to other critical structures or organs of the body, and the extent to which the tumor has spread.

As used herein, the term “composition” is intended to encompass a product comprising the specified ingredients in the specific amounts, as well as any product which results, directly or indirectly, from combination of the specific ingredients in the specified amounts.

A pharmaceutical composition which is useful for the treatment of cancer in the instant invention may comprise, one or more PSA conjugates, in combination with pharmaceutically acceptable carriers, excipients or diluents, according to standard pharmaceutical practice. The composition may be administered to mammals, preferably humans. The composition can be administered orally or parenterally, including the intravenous, intramuscular, intraperitoneal, subcutaneous, rectal and topical routes of administration.

A pharmaceutical composition of the instant invention may comprise one or more PSA conjugates in combination. Also preferably, these agents are in combination with pharmaceutically acceptable carriers, excipients or diluents, according to standard pharmaceutical practice. The composition may be administered to mammals, preferably humans. The composition can be administered by oral, parenteral (e.g., intramuscular, intraperitoneal, intravenous or subcutaneous injection, or implant), nasal, vaginal, rectal, sublingual, or topical routes of administration and can be formulated in dosage forms appropriate for each route of administration.

Preferably the compositions according to the present invention are in unit dosage forms such as tablets, pills, capsules, powders, granules, solutions or suspensions, or suppositories, for oral, parenteral or rectal administration, by inhalation or insufflation or administration by trans-dermal patches or by buccal cavity absorption wafers.

For preparing solid compositions such as tablets, the principal active ingredient(s) is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil, peanut oil or soybean oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

Preferred compositions for administration by injection include those comprising a PSA conjugate as the active ingredient, in association with a surface-active agent (or wetting agent or surfactant) or in the form of an emulsion (as a water-in-oil or oil-in-water emulsion).

Suitable surface-active agents include, in particular, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5% surface-active agent, and preferably between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™. Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion will preferably comprise fat droplets between 0.1 and 1.0 μm, particularly 0.1 and 0.5 μm, and have a pH in the range of 5.5 to 8.0.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral or nasal respiratory route for local or systemic effect. Compositions in preferably sterile pharmaceutically acceptable solvents may be nebulised by use of inert gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

Compositions of the present invention may also be presented for administration in the form of trans-dermal patches using conventional technology. The compositions may also be administered via the buccal cavity using, for example, absorption wafers.

Compositions in the form of tablets, pills, capsules or wafers for oral administration are particularly preferred.

The pharmaceutical compositions containing the active ingredients may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions useful in the instant methods of treatment may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The instant compositions may also be administered in the form of a suppositories for rectal administration of the drug. These compositions can be prepared by mixing the instant composition with a suitable non-irritating excipient which is solid at ordinary temperatures but liquid at the rectal temperature and will therefore melt in the rectum to release the composition. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the PSA conjugate(s) are employed. (For purposes of this application, topical application shall include mouth washes and gargles.) The compositions useful in the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The composition of a PSA conjugate(s) may also be co-administered with other well known therapeutic agents that are selected for their particular usefulness against the condition that is being treated.

The instant method of treatment may also be combined with surgical treatment (such as surgical removal of tumor and/or prostatic tissue) where appropriate. The above descriptions of pharmaceutical compositions are also applicable to pharmaceutical compositions of the individual agents if they are administered separately according to the invention.

If formulated as a fixed dose, the compositions useful in the instant invention employ the PSA conjugate(s) within the dosage ranges described below.

When compositions according to this invention are administered into a human subject, the daily dosage will normally be determined by the prescribing physician with the dosage generally varying according to the age, weight, and response of the individual patient, as well as the severity of the patient's symptoms.

The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease or disorder, the patient's weight, special diets then being followed by a patient, concurrent medication, the bioavailability upon oral administration of the compound and other factors which those skilled in the art will recognize.

In the treatment of a condition in accordance with the present invention, an appropriate dosage level will generally be about 0.01 μg to 50 mg per kg patient body weight per day which may be administered in single or multiple doses. Preferably, the dosage level will be about 0.1 μg to about 25 mg/kg per day; more preferably about 0.5 μg to about 10 mg/kg per day. A compound may be administered on a regimen of several times per day, for example 1 to 4 times per day, preferably once or twice per day. When using an injectable formulation, a suitable dosage level is about 0.1 μg to 10 mg/kg per day, preferably about 0.5 μg to 5 mg/kg per day, and especially about 1 μg to 1 mg/kg per day.

Pharmaceutical compositions of the present invention may be provided in a solid dosage formulation preferably comprising about 100 μg to 500 mg active ingredient, more preferably comprising about 100 μg to 250 mg active ingredient. The pharmaceutical composition is preferably provided in a solid dosage formulation comprising about 100 μg, 1 mg, 5 mg, 10 mg, 25 mg, 50 mg, 100 mg, 200 mg or 300 mg active ingredient. Administration of the PSA conjugate occurs in an amount between about 10 mg/m² of body surface area to about 4 g/m² of body surface per day, preferably between about 50 mg/m² of body surface to about 3 g/m² of body surface per day.

It will be appreciated that the amount of the composition of the instant invention required for use in treating cancer in a patient will vary not only with the particular compounds or compositions selected but also with the route of administration, the nature of the condition being treated, and the age and condition of the patient, and will ultimately be at the discretion of the patient's physician or pharmacist. The length of time during which the instant composition will be given varies on an individual basis.

It will be appreciated by those skilled in the art that reference herein to treatment extends to prophylaxis (prevention) as well as the treatment of the noted diseases/disorders and symptoms. Because the specific diagnosis of chronic nonbacterial prostatitis or prostatodynia in a particular patient may be difficult, the patient may benefit from the prophylactic administration of a subject compound in accordance with the present invention.

Examples are provided for the purpose of further illustration only and are not intended to be limitations on the disclosed invention.

The starting materials and reagents for the subject processes are either commercially available or are known in the literature or may be prepared following literature methods described for analogous compounds. The skills required in carrying out the reaction and purification of the resulting reaction products are known to those in the art. Purification procedures include crystallization, distillation, normal phase or reverse phase chromatography.

The following examples are provided for the purpose of further illustration only and are not intended to be limitations on the disclosed invention.

### EXAMPLE 1

Preparation of [N-Ac-(4-trans-L-Hyp)]-Ala-Ser-Chg-Gln-Ser-Leu-Dox

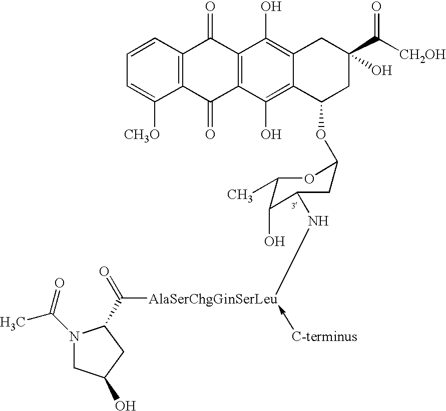

Step A:

[N-Ac-(4-trans-L-Hyp(Bzl))] -Ala-Ser(Bzl)Chg-Gln-Ser(Bzl)Leu-PAM Resin (1-1).

Starting with 0.5 mmol (0.67g) Boc-Leu-PAM resin, the protected peptide was synthesized on a 430A ABI peptide synthesizer. The protocol used a 4 fold excess (2 mmol) of each of the following protected amino acids: Boc-Ser(Bzl), Boc-Gln, Boc-Chg, Boc-Ala, N-Boc-(4-trans-L-Hyp(Bzl)). Coupling was achieved using DCC and HOBT activation in methyl-2-pyrrolidinone. Acetic acid was used for the introduction of the N terminal acetyl group. Removal of the Boc group was performed using 50% TFA in methylene chloride and the TFA salt neutralized with diisopropylethylamine. At the completion of the synthesis the peptide resin was dried to yield Intermediate 1-1.

Step B:

[N-Ac-(4-trans-L-Hyp)]-Ala-Ser-Chg-Gln-Ser-Leu-OH (1-2)

The protected peptide resin (1-1), 1.2 g, was treated with HF (20 ml) for 1 hour at 0° C. in the presence of anisole (2 ml). After evaporation of the HF, the residue was washed with ether, filtered and extracted with H₂O (200 ml). The filtrate was lyophilyzed to yield Intermediate 1-2.

Step C:

[N-Ac-(4-trans-L-Hyp)]-Ala-Ser-Chg-Gln-Ser-Leu-Dox

The above described intermediate (1-2), 1.157 g (1.45 mmol) was dissolved in DMSO (30 ml) and diluted with DMF (30 ml). To the solution was added doxorubicin hydrochloride, 516 mg (0.89 mmol) followed by 0.310 ml of diisopropylethylamine (1.78 mmol). The stirred solution was cooled (0° C.) and 0.276 ml of diphenylphosphoryl azide (1.28 mmol) added. After 30 minutes, an additional 0.276 ml (1.28 mmol) of DPPA was added and the pH adjusted to ˜7.5 (pH paper) with diisopropylethylamine (DIEA). The pH of the cooled reaction (0° C.) was maintained at ˜7.5 with DIEA for the next 3 hours and the reaction stirred at 0-4° C. overnight. After 18 hours, the reaction (found to be complete by analytical HPLC, system A) was concentrated to an oil. Purification of the crude product was achieved by preparative HPLC, Buffer A=0.1% NH₄OAc-H₂O; B=CH₃CN. The crude product was dissolved in 400 ml of 100% A buffer, filtered and purified on a C-18 reverse phase HPLC radial compression column (Waters, Delta-Pak, 15μM, 100 Å). A step gradient of 100% A to 60% A was used at a flow rate of 75 ml/min (UV=214 nm). Homogeneous product fractions (evaluated by HPLC, system A) were pooled and freeze-dried. The product was dissolved in H₂O (300 ml), filtered and freeze-dried to provide the purified title compound.

Physical Properties

The physical/chemical properties of the product of Step C are shown below:

| Molecular Formula: | C₆₂H₈₅N₉O₂₃ | |
|---|---|---|
| Molecular Weight: | 1323.6 | |
| High Resolution ES Mass Spec: | 1341.7 (NH₄⁺) | |
| UPLC: | System A | |
| Column: | Vydac 15 cm #218TP5415, C18 | |
| Eluant: | Gradient 95:5 (A:B) to 5:95 (A:B) over | |
| | 45 min. A = 0.1% TFA/H₂O, B = 0.1% | |
| | TFA/Acetonitrile | |
| Flow: | 1.5 ml/min. | |
| Wavelength: | 214 nm, 254 nm | |
| Retention Time: | 18.2 min. | |
| Amino Acid Compositional | Theory | Found |
| Analysis^{1:} | Ala (1) | 1.00 |
| | Ser (2) | 1.88 |
| | Chg (1) | 0.91 |
| | Gln² (1) | 1.00 (as Glu) |
| | Hyp (1) | 0.80 |
| | Leu (1) | 1.01 |
| | Peptide Content: | 0.657 μmol/mg |

Note:
¹20 hr., 100° C., 6N HCl
²Gln converted to Glu

### EXAMPLE 2

Preparation of [N-Glutaryl-(4-trans-L-Hyp)]-Ala-Ser-Chg-Gln-Ser-Leu-Dox (Compound B)

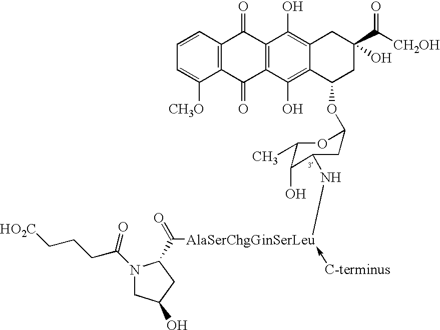

Step A:

[N-Glutaryl(OFm)-(4-trans-L-Hyp)]-Ala-Ser-Chg-Gln-Ser-Leu-PAM Resin

Starting with 0.5 mmol (0.67g) Boc-Leu-PAM resin, the protected peptide was synthesized on a 430A ABI peptide synthesizer. The protocol used a 4 fold excess (2 mmol) of each of the following protected amino acids: Fmoc-Ser(tBu), Fmoc-Gln(Trt), Fmoc-Chg, Fmoc-Ala, Boc-(4-trans-L-Hyp). Coupling was achieved using DCC and HOBT activation in methyl-2-pyrrolidinone. The intermediate mono fluorenylmethyl ester of glutaric acid [Glutaryl(OFm)] was used for the introduction of the N-terminal glutaryl group. Removal of the Fmoc group was performed using 20% piperidine. The acid sensitive protecting groups, Boc, Trt and tBu, were removed with 50% TFA in methylene chloride. Neutralization of the TFA salt was with diisopropylethylamine. At the completion of the synthesis, the peptide resin was dried to yield the title compound.

Step B:

[N-Glutaryl(OFm)-(4-trans-L-Hyp)]-Ala-Ser-Chg-Gln-Ser-Leu-OH

The protected peptide resin from Step A, 1.2 g, was treated with Hf (20 ml) for 1 hr at 0° C. in the presence of anisole (2 ml). After evaporation of the HF, the residue was washed with ether, filtered and extracted with DMF. The DMF filtrate (75 ml) was concentrated to dryness and triturated with H₂O. The insoluble product was filtered and dried to provide the title compound.

Step C:

[N-Glutaryl(OFm)-(4-trans-L-Hyp)]-Ala-Ser-Chg-Gln-Ser-Leu-Dox

The above prepared intermediate from Step B, (1.33g, 1.27 mmol) was dissolved in DMSO (6 ml) and DMF (69 ml). To the solution was added doxorubicin hydrochloride, 599 mg (1.03 mmol) followed by 376%1 of diisopropylethylamine (2.16 mmol). The stirred solution was cooled (0° C.) and 324 μl of diphenylphosphoryl azide (1.5 mmol) added. After 30 minutes, an additional 324 μl of DPPA was added and the pH adjusted to −7.5 (pH paper) with diisopropylethylamine (DIEA). The pH of the cooled reaction (0° C.) was maintained at −7.5 with DIEA for the next 3 hours and the reaction stirred at 0-4° C. overnight. After 18 hours, the reaction (found to be complete by analytical HPLC, system A) was concentrated to provide the title compound as an oil.

Step D:

[N-Glutaryl-(4-trans-L-Hyp)]-Ala-Ser-Chg-Gln-Ser-Leu-Dox

The above product from Step C was dissolved in DMF (54 ml), cooled (0° C.) and 14 ml of piperidine added. The solution was concentrated to dryness and purified by preparative HPLC. (A=0.1% NH₄OAc-H₂O; B=CH₃CN.) The crude product was dissolved in 100 ml of 80% A buffer, filtered and purified on a C-18 reverse phase HPLC radial compression column (Waters, Delta-Pak, 15 μ, 100 Å). A step gradient of 80% A to 67% A was used at a flow rate of 75 ml/min (uv=214 nm). Homogeneous product fractions (evaluated by HPLC, system A) were pooled and freeze-dried. The product was further purified using the above HPLC column. Buffer A=15% acetic acid-H₂O; B=15% acetic acid-methanol. The product was dissolved in 100 ml of 20% B/80% A buffer and purified. A step gradient of 20% B to 80% B was used at a flow rate of 75 ml/min (uv=260 nm). Homogeneous product fractions (evaluated by HPLC, system A) were pooled, concentrated and freeze-dried from H₂O to yield the purified title compound.

| | |
|---|---|
| High Resolution ES Mass Spec: | 1418.78 (Na⁺) |
| HPLC: | System A |
| Column: | Vydac 15 cm #218TP5415, C18 |
| Eluant: | Gradient 95:5 (A:B) to 5:95 (A:B) |
| | over 45 min. |
| | A = 0.1% TFA/H₂O, |
| | B = 0.1% TFA/Acetonitrile |
| Flow: | 1.5 ml/min. |
| Wavelength: | 214 nm, 254 nm |
| Retention Time: | 18.3 min. |

Amino Acid Compositional Analysis¹:

| | |
|---|---|
| Theory | Found |
| Ala(1) | 0.99 |
| Ser(2) | 2.02 |
| Chg(1) | 1.00 |
| Gln² (1) | 1.01 (as Glu) |
| Hyp(1) | 0.99 |
| Leu(1) | 1.00 |
| Peptide Content: | 0.682 μmol/mg |

Note:
¹20 hr., 100° C., 6N HCl
²Gln converted to Glu

### EXAMPLE 3

Preparation of (4-trans-L-Hyp)-Ala-Ser-Chg-Gln-Ser-Leu-Dox

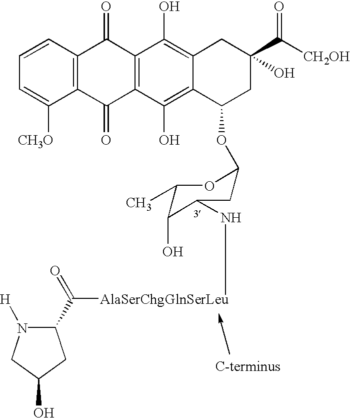

Step A:

Fmoc-(4-trans-L-Hyp(Bzl))-Ala-Ser(Bzl)Chg-Gln-Ser(Bzl)Leu-PAM Resin

Starting with 0.5 mmol (0.67g) Boc-Leu-PAM resin, the protected peptide was synthesized on a 430A ABI peptide synthesizer. The protocol used a 4 fold excess (2 mmol) of each of the following protected amino acids: Boc-Ser (Bzl), Boc-Gln, Boc-Chg, Boc-Ala, N-Boc-(4-trans-L-Hyp(Bzl)). Coupling was achieved using DCC and HOBT activation in methyl-2-pyrrolidinone. Fmoc-OSu (succinamidyl ester of Fmoc) was used for the introduction of the N-terminal protecting group. Removal of the Boc group was performed using 50% TFA in methylene chloride and the TFA salt neutralized with diisopropylethylamine. At the completion of the synthesis the peptide resin was dried to yield the title intermediate.

Step B:

Fmoc-(4-trans-L-Hyp)-Ala-Ser-Chg-Gln-Ser-Leu-OH

The protected peptide resin from Step A, 1.1 g, was treated with HF (20 ml) for 1 hour at 0° C. in the presence of anisole (2 ml). After evaporation of the HF, the residue was washed with ether, filtered and extracted with H₂O (200 ml). The filtrate was lyophilyzed to yield the title intermediate.

Step C:

Fmoc-(4-trans-L-Hyp)-Ala-Ser-Chg-Gln-Ser-Leu-Dox

The intermediate from Step B, 0.274 g, was dissolved in DMSO (10 ml) and diluted with DMF (10 ml). To the solution was added doxorubicin hydrochloride, 104 mg followed by 62 μL of diisopropylethylamine (DIEA). The stirred solution was cooled (0° C.) and 56 μL of diphenylphosphoryl azide added. After 30 minutes, an additional 56 μL of DPPA was added and the pH adjusted to ˜7.5 (pH paper) with DIEA. The pH of the cooled reaction (0° C.) was maintained at ˜7.5 with DIEA. After 4 hours, the reaction (found to be complete by analytical HPLC, system A) was concentrated to an oil. HPLC conditions, system A.

Step D:

(4-trans-L-Hyp)-Ala-Ser-Chg-Gln-Ser-Leu-Dox

The above product from Step C was dissolved in DMF (10 ml), cooled (0° C.) and 4 ml of piperidine added. The solution was concentrated to dryness and purified by preparative HPLC. (A=0.1% NH₄OAc-H₂O; B═CH₃CN.) The crude product was dissolved in 100 ml of 90% A buffer, filtered and purified on a C-18 reverse phase HPLC radial compression column (Waters, Delta-Pak, 15 μg, 15 μ, 100 Å). A step gradient of 90% A to 65% A was used at a flow rate of 75 ml/min (uv=214 nm). Homogeneous product fractions (evaluated by HPLC, system A) were pooled and freeze-dried.

| | |
|---|---|
| Molecular Formula: | C₆₀H₈₃N₉O₂₂ |
| Molecular Weight: | 1281.56 |
| High Resolution ES Mass Spec: | 1282.59 (MH⁺) |
| HPLC: | System A |
| Column: | Vydac 15 cm #218TP5415,C18 |
| Eluant: | Gradient 95:5 (A:B) to 5:95 (A:B) |
| | over 45 min. |
| | A = 0.1% TFA/H₂O, |
| | B = 0.1% TEA/Acetonitrile |
| Flow: | 1.5 ml/mm. |
| Wavelength: | 214 nm, 254 nm |
| Retention Time: | 17.6 min. |

Amino Acid Compositional Analysis¹:

| | |
|---|---|
| Theory | Found |
| Ala(1) | 1.00 |
| Ser(2) | 1.94 |
| Chg (1) | 0.94 |
| Gln² (1) | 1.05 (as Glu) |
| Hyp(1) | 0.96 |
| Leu(1) | 1.03 |
| Peptide Content: | 0.690 μmol/mg |

Note:
¹20 hr., 100° C., 6N HCl
²Gln converted to Glu

### EXAMPLE 4

des-Acetylvinblastine-4-O-(N-Acetyl-4-trans-L-Hyp-Ser-Ser-Chg-Gln-Ser-Ser-Pro) ester

Step A:

Preparation of 4-des-Acetylvinblastine

A sample of 2.40g (2.63 mmol) of vinblastine sulfate (Sigma V-1377) was dissolved under N₂ in 135 ml of absolute methanol and treated with 45 ml of anhydrous hydrazine, and the solution was stirred at 20-25° C. for 18 hours. The reaction was evaporated to a thick paste, which was partitioned between 300 ml of CH₂Cl₂ and 150 ml of saturated NaHCO₃. The aqueous layer was washed with 2 100-ml portions of CH₂Cl₂, and each of the 3 CH₂Cl₂ layers in turn was washed with 100 ml each of H₂O (2×) and saturated NaCl (1×). The combined organic layers were dried over anhydrous Na₂SO₄, and the solvent was removed at reduced pressure to yield the title compound as an off-white crystalline solid. This material was stored at −20° C. until use.

Step B:

Preparation of 4-des-Acetylvinblastine 4—O-(Prolyl) ester

A sample of 804 mg (1.047 mmol) of 4-des-acetylvinblastine, dissolved in 3 ml of CH₂Cl₂ and 18 ml of anhydrous pyridine under nitrogen, was treated with 1.39 g of Fmoc-proline acid chloride (Fmoc-Pro-Cl, Advanced Chemtech), and the mixture was stirred for 20 hours at 25° C. When analysis by HPLC revealed the presence of unreacted starting des-acetylvinblastine, another 0.50 g of Fmoc-Pro-Cl was added, with stirring another 20 hours to complete the reaction. Water (ca. 3 ml) was added to react with the excess acid chloride, and the solution was then evaporated to dryness and partitioned between 300 ml of EtOAc and 150 ml of saturated NaHCO₃, followed by washing twice with saturated NaCl. After drying (Na₂SO₄), the solvent was removed under reduced pressure to give an orange-brown residue, to which was added 30 ml of DMF and 14 ml of piperidine, and after 5 minutes the solution was evaporated under reduced pressure to give a orange-yellow semi-solid residue. After drying in vacuo for about 1 hour, approx. 200 ml of H₂O and 100 ml of ether was added to this material, followed by glacial HOAc dropwise with shaking and sonication until complete dissolution had occurred and the aqueous layer had attained a stable pH of 4.5-5.0 (moistened pH range 4-6 paper). The aqueous layer was then washed with 1 100-ml portion of ether, and each ether layer was washed in turn with 50 ml of H₂O. The combined aqueous layers were subjected to preparative HPLC in 2 portions on a Waters C₄ Delta-Pak column 15 μM 300A (A=0.1% TFA/H₂O; B=0.1% TFA/CH₃CN), gradient elution 95·70% A/70 min. Pooled fractions yielded, upon concentration and lyophilization, the title compound.

Step C:

N-Acetyl-4-trans-L-Hyp-Ser-Ser-Chg-Gln-Ser-Ser-WANG Resin

Starting with 0.5 mmole (0.61 g) of Fmoc-Ser(t-Bu)-WANG resin loaded at 0.82 mmol/g, the protected peptide was synthesized on a ABI model 430A peptide synthesizer adapted for Fmoc/t-butyl-based synthesis. The protocol used a 2-fold excess (1.0 mmol) of each of the following protected amino acids: Fmoc-Ser (t-Bu)-OH, Fmoc-Gln-OH, Fmoc-Chg-OH, Fmoc-4-trans-L-Hyp-OH; and acetic acid (double coupling). During each coupling cycle Fmoc protection was removed using 20% piperidine in N-methyl-2-pyrrolidinone (NMP), followed by washing with NMP. Coupling was achieved using DCC and HOBt activation in NMP. At the completion of the synthesis, the peptide resin was dried to yield the title compound.

Step D:

N-Acetyl-4-trans-L-Hyp-Ser-Ser-Chg-Gln-Ser-Ser-OH

One 0.5-mmol run of the above peptide-resin was suspended in 25 ml of TFA, followed by addition of 0.625 ml each of H₂O and triisopropylsilane, then stirring at 25° for 2.0 hours. The cleavage mixture was filtered, the solids were washed with TFA, the solvents were removed from the filtrate under reduced pressure, and the residue was triturated with ether to give a pale yellow solid, which was isolated by filtration and drying in vacuo to afford the title compound.

HPLC conditions, system A:

| | |
|---|---|
| Column: | Vydac 15 cm #218TP5415, C18 |
| Eluant: | Gradient (95%A · 50%A) over 45 min. |
| | A = 0.1% TFA/H₂O, B = 0.1% |
| | TFA/acetonitrile |
| Flow.: | 1.5 ml/min. |

High Resolution ES/FT-MS: 789.3

Step E:

des-Acetylvinblastine-4—O-(N-Acetyl-4-trans-L-Hyp-Ser-Ser-Chg-Gln-Ser-Ser-Pro) ester

Samples of 522 mg (0.66 mmol) of the peptide prepared as described in step D and 555 mg (ca. 0.6 mmol) of 4-des-Acetylvinblastine 4—O-(Prolyl) ester from Step B, prepared as above, were dissolved in 17 ml of DMF under N₂. Then 163 mg (1.13 mmol) of 1-hydroxy-7-azabenzotriazole (HOAt) was added, and the pH was adjusted to 6.5-7 (moistened 5-10 range pH paper) with 2,4,6-collidine, followed by cooling to 0° C. and addition of 155 mg (0.81 mmol) of 1-(3-dimethyl-aminopropyl)-3-ethylcarbodiimide hydrochloride (EDC). Stirring was continued at 0-5° C. until completion of the coupling as monitored by analytical HPLC (A=0.1% TFA/H₂O; B=0.1% TFAICH₃CN), maintaining the pH at 6.5-7 by periodic addition of 2,4,6-collidine. After 12 hours the reaction was worked up by addition of ˜4 ml of H₂O and, after stirring 1 hour, concentrated to a small volume in vacuo and dissolution in ca. 150 ml of 5% HOAc. and preparative HPLC in two portions on a Waters C₁₈ Delta-Pak column 15 μM 300A (A=0.1% TFA/H₂O; B=0.1% TFA/CH₃CN, gradient elution 95·65% A/70 min). Homogeneous fractions containing the later-eluting product (evaluated by HPLC, system A, 95·65% A/30 min) from both runs were pooled and concentrated to a volume of ˜50 ml and passed through approx. 40 ml of AG4X4 ion exchange resin (acetate cycle), followed by freeze-drying to give the title compound as a lyophilized powder.

High Resolution ES/FT-MS: 1637.0

### EXAMPLE 5

des-Acetylvinblastine-4-O-(N-Acetyl-4-trans-L-Hyp-Ser-Ser-Chg-Gln-Ser-Ser-Pro) ester acetate

A sample of 4.50 g (3.7 mmol) of 4—O-(prolyl) des-acetylvinblastine TFA salt, prepared as described in Example 4, Step B, was dissolved in 300 ml of DMF under N₂, and the solution was cooled to 0° C. Then 1.72 g (10.5 mmol) of 3,4-dihydro-3-hydroxy-4-oxo-1,2,3-benzotriazine (ODHBT) was added, and the pH was adjusted to 7.0 (moistened 5-10 range pH paper) with N-methylmorpholine (NMM), followed by the addition of 4.95 g (5.23 mmol) of the N-acetyl-heptapeptide of Example 4, Step D, portionwise allowing complete dissolution between each addition. The pH was again adjusted to 7.0 with NMM, and 1.88 g (9.8 mmol) of 1-(3-dimethylaminopropyl)-3-ethylcarbodiimide hydrochloride (EDC) was added, followed by stirring of the solution at 0-5° C. until completion of the coupling as monitored by analytical HPLC (system A), maintaining the pH at ca. 7 by periodic addition of NMM. The analysis showed the major component at 26.3 minutes retention time preceded by a minor component (ca. 10%) at 26.1 min, identified as the D-Ser isomer of the title compound. After 20 hours the reaction was worked up by addition of 30 ml of H₂O and, after stirring 1 hour, concentrated to a small volume in vacuo and dissolution in ca. 500 ml of 20% HOAc. and preparative HPLC in 12 portions on a Waters C₁₈ Delta-Pak column 15 mM 300A (A=0.1% TFA/H₂O; B =0.1% TFA/CH₃CN), gradient elution 85·65% A/90 min) at a flow rate of 80 ml/min.

Homogeneous fractions (evaluated by HPLC, system C) representing approx. one-fourth of the total run were pooled and concentrated to a volume of ˜150 ml and passed through approx. 200 ml of Bio-Rad AG4×4 ion exchange resin (acetate cycle), followed by freeze-drying of the eluant gave the acetate salt of the title compound as a lyophilized powder: retention time (system A) 26.7 minutes, 98.9% pure; high resolution ES/FT-MS m/e 1636.82; amino acid compositional analysis 20 hours, 100° C., 6N HCl (theory/found), Ser4/3.91 (corrected), Glu 1/0.92 (Gln converted to Glu), Chg 1/1.11,Hyp 1/1.07, Pro 1/0.99, peptide content 0.516 mmol/mg.

Further combination of homogeneous fractions and purification from side fractions, processing as above through approx. 500 ml of ion exchange resin, afforded an additional amounts of the title compound.

| | |
|---|---|
| HPLC conditions, | |
| system A: | |
| Column: | Vydac 15 cm #218TP5415, C18 |
| Flow: | 1.5 ml/min. |
| Eluant: | Gradient (95%A · 50%A) over 45 min. |
| | A = 0.1% TFA/H₂O, B = 0.1% TFA/acetonitrile |
| Wavelength: | 214 nm, 280 nm |
| HPLC conditions, | |
| system C: | |
| Column: | Vydac 15 cm #218TP5415, C18 |
| Flow: | 1.5 ml/min. |
| Eluant: | Gradient (85%A · 65%A) over 30 min. |
| | A = 0.1% TFA/H₂O, B = 0.1% TFA/acetonitnle |
| Wavelength: | 214 nm, 280 nm |

### EXAMPLE 6

Preparation of 4-des-Acetylvinblastine-23-(4′-aminomethylbicyclo-[2.2.2] octane)methylamide (BDAM-(dAc)vinblastine)

Step A:

Preparation of 4-des-Acetylvinblastine-23-hydrazide

A sample of 3.99 g (4.38 mmol) of vinblastine sulfate (Sigma V-1377) was dissolved in 30.4 ml of 1:1 (v/v) absolute ethanol/anhydrous hydrazine, under N₂, and the solution was heated in an oil bath at 60-65° C. for 23 hours. Upon cooling, the solution was evaporated to a thick paste, which was partitioned between 300 ml of CH₂Cl₂ and 150 ml of saturated NaHCO₃. The aqueous layer was washed with 2 100-ml portions of CH₂Cl₂, and each of the 3 CH₂C₁₂ layers in turn was washed with 100 ml each of H₂O (2×) and saturated NaCl (1×). The combined organic layers were dried over anhydrous Na₂SO₄, and the solvent was removed in vacuo to yield, after drying 20 hours in vacuo, the title compound as a white crystalline solid. This material was dissolved in 82 ml of dry, degassed DMF for storage at −20° C. until use (conc. 36 mg/ml).

Step B:

Boc-4-aminomethylbicvclo-[2.2.2]octane carboxylic acid

A sample of 8.79 g (40.0 mmol) of 4-carboxybicyclo-[2.2.2] octanemethylamine hydrochloride salt suspended in 100 ml each of THF and H₂O was treated with 20.0 ml (14.6 g=3.3 equiv.) of TEA, followed by 11.8 g (47.9 mmol) of BOC—ON reagent. All went into solution, and after stirring 24 hours the solution was concentrated in vacuo to a volume of about 50 ml and partitioned between 100 ml of ether and 300 ml of H₂O. After addition of about 2 ml of TEA the aqueous layer was washed with ether (3×), each ether in turn washed with H₂O, and the combined aqueous layer was acidified with 5% KHSO₄ to give the title compound as a white solid, isolated by filtration and drying in vacuo.

Step C:

Boc-4-aminomethylbicyclo-[2.2.21]octane carboxamide

A stirred solution under N₂ of 12.0 g (42.5 mmol) of the product from step B in 100 ml of DMF was treated with 8.0 g (49.3 mmol) of carbonyl-diimidazole. After 30 minutes the DMF was evaporated in vacuo to afford 50-60 ml of a light brown paste, which was stirred and treated with 70 ml of conc. NH₄OH rapidly added. The initial solution turned to a white paste within 30 minutes, after which H₂O was added up to a total volume of 400 ml to complete precipitation of product, which was triturated and isolated by filtration and washing with H₂O, and dried in vacuo to yield the title compound as a white solid.

Step D:

Boc-4-aminomethylbicyclo-[2.2.2]octane nitrile

A solution of 7.52 g (26.6 mmol) of the product from step C in 50 ml of CH₂Cl₂ and 80 ml of anhydrous pyridine was treated with 11.12 g of (methoxycarbonylsulfamoyl)-triethyl-ammonium hydroxide inner salt (Burgess reagent) in 1-g portions over 5 minutes. After stirring for 1.5 hours, TLC (90-10-1, CHCl₃—CH₃OH—H₂O) showed complete conversion to product, and the solution was evaporated to give a paste, to which H₂O was added, up to 400 ml, with trituration and stirring to afford, after standing 20 hours at 0° C., filtration and drying in vacuo, the title compound as a white solid. Step E: Boc-4-aminomethylbicyclo-[2.2.2]octane methylamine A solution of 6.75 g (25.5 mmol) of the product from step D in 200 ml of CH₃₀H plus 4 ml of HOAc and 2 ml of H₂O was hydrogenated over 1.63 g of PtO₂ in a Parr shaker at 55 psi for 22 hours. The catalyst was removed by filtration through Celite, and the filtrate was concentrated in vacuo to an oily residue, which was flushed/evaporated with CH₃₀H (1×) and CH₂Cl₂ (2×). Product began to crystallize toward the end of the evaporation, and ether (up to 300 ml) was added to complete the precipitation. The white solid was triturated and isolated by filtration and washing with ether to give, after drying in vacuo, the title compound as the acetate salt.

400 Mhz ¹H-NMR (CDCl₃): δ (ppm, TMS) 4.5 (Is, Boc-NH); 2.9 (2br d, —CH₂—NH-Boc); 2.45 (2br s, —CH₂—NH₂); 2.03 (3s, CH₃COOH);1.45 (9s, Boc); 1.40 (12s, ring CH₂).

Step F:

Preparation of 4-des-Acetylvinblastine-23-(4′-aminomethylbicyclo-[2.2.2]octane) methylamide (BDAM-(dAc)vinblastine)

A 30-ml aliquot of the above DMF solution of 4-des-acetylvinblastine-23-hydrazide (1.41 mmol), cooled to −15° C. under Argon, was converted to the azide in situ by acidification with 4M HCl in dioxane to pH<1.5 (moistened 0-2.5 range paper), followed by addition of 0.27 ml (1.3 equiv) of isoamyl nitrite and stirring for 1 hour at 10-15° C. The pH was brought to 7 by the addition of DIEA, and a slurry of 1.27 g (3.8 mmol) of the Boc diamine product from step E above in 20 ml of DMF was then added, and the reaction was allowed to warm slowly to 15-20° C. over 2 hours, at which point coupling was complete, as monitored by analytical HPLC (A=0.1% TFA/H₂O; B=0.1% TFA/CH₃CN). The solvent was removed in vacuo and the residue partitioned between EtOAc and 5% NaHCO₃, the organic layer washed with 5% NaCl, and the aqueous layers back-extracted with CH₂Cl₂ to assure removal of the intermediary Boc-BDAM-(dAc)vinblastine. The combined organic layers were dried over Na₂SO₄, the solvent was removed under reduced pressure, and the residue, after flush/evaporation twice from CH₂Cl₂, was dissolved in 30 ml of CH₂Cl₂ and treated with 30 ml of TFA for 30 minutes. The solvents were rapidly removed in vacuo, and the residue was dissolved in 300 ml of 10% HOAc for purification by preparative HPLC in 5 portions on a Waters C₄ Delta-Pak column 15 μM 300A (A=0.1% TFA/H₂O; B=0.1% TFA/CH₃CN), gradient elution 95·70% A/60 min, isocratic 70%/20 min. Homogeneous fractions (evaluated by HPLC, system A, 95˜50% A) from the five runs were pooled and concentrated in acuo, followed by freeze-drying to give of the title compound as the lyophilized TFA salt.

| | |
|---|---|
| HPLC conditions, | |
| system A: | |
| Column: | Vydac 15 cm #218TP5415, C18 |
| Eluant: | Gradient (A · B) over 45 min. |
| | A = 0.1% TFA/H₂O, B = 0.1% TFA/acetonitrile |
| Flow: | 1.5 ml/min. |

Retention time: BDAM (dAc) vinblastine 23.5 min. (95% 50% A) 97% purity

High Resolution ES/FT-MS: 905.63

Compound content by elemental analysis=0.714 μmol/mg: N (calc)=9.28 N (found)=6.00

### EXAMPLE 7

Preparation of 4-des-Acetylvinblastine-23-(N-Acetyl-Ser-Ser-Ser-Chg-Gln-Ser-Val-BDAM) amide acetate salt

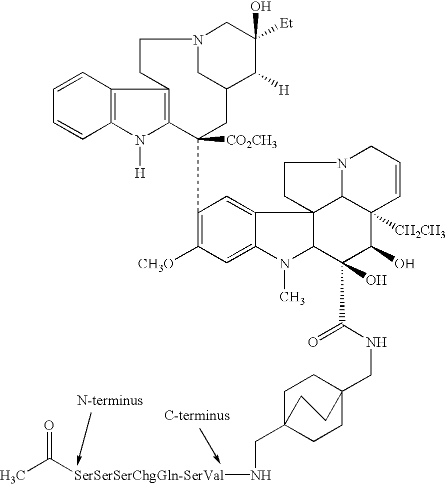

Step A:

N-Acetyl-Ser-Ser-Ser-Chg-Gln-Ser-Val-PAM Resin

Starting with 0.5 mmole (0.68 g) of Boc-Val-PAM resin, the protected peptide was synthesized on a ABI model 430A peptide synthesizer. The protocol used a 4-fold excess (2.0 mmol) of each of the following protected amino acids: Boc-Ser(Bzl)-OH, Boc-Gln-OH, Boc-Chg-OH; and acetic acid (2 couplings). During each coupling cycle Boc protection was removed using TFA, followed by neutralization with DIEA. Coupling was achieved using DCC and HOBt activation in N-methyl-2-pyrrolidinone. At the completion of the synthesis, the peptide resin was dried to yield the title compound.

Step B:

N-Acetyl-Ser-Ser-Ser-Chg-Gln-Ser-Val-OH

Three 0.5-mmol runs of the above peptide-resin (3.5 g) were combined and treated with liquid HF (65 ml) for 1.5 hours at 0° C. in the presence of anisole (6 ml). After evaporation of the HF, the residue was washed with ether, filtered and leached with 150 ml of DMF in several portions, adding DIEA to pH ˜8, followed by removal of the DMF in vacuo to a volume of 100 ml. The concentration was determined as ca. 11.7 mg/ml (by weighing the dried resin before and after leaching.

The sample purity was determined as 96% by HPLC. The solution was used directly for conjugation with BDAM-(dAc) vinblastine.

Step C:

4-Des-acetylvinblastine-23-(N-Acetyl-Ser-Ser-Ser-Chg-Gln-Ser-Val-BDAM) amide acetate salt

To 58 ml (equivalent to 0.875 mmol of peptide) of the solution from step B was added 530 mg (0.520 mmol) of BDAM-(dAc)vinblastine, prepared as described in Example 6, Step F, under N₂, cooling to 0° C., and the pH was adjusted to ˜8 (moistened 5-10 range pH paper) with DIEA. Then 0.134 ml (0.62 mmol) of DPPA was added, followed by stirring at 0-5° C. until completion of the coupling as monitored by analytical HPLC (A=0.1% TFA/H₂O; B=0.1% TFA/CH₃CN), maintaining the pH at >7 by periodic addition of DIEA. After 24 hours, the reaction was worked up by addition of 10 ml of H₂O, stirring 1 hour and concentration to small volume in vacuo, then dissolution in ca. 100 ml of 10% HOAc/5% CH₃CN, adjustment of the pH to 5 with NH₄HCO₃, filtration to remove insolubles, and preparative HPLC in 3 portions on a Waters C₄ Delta-Pak column 15RM 300A (A=0.1% NH₄HCO₃/H₂O; B=CH₃CN), gradient elution 95·40% A/70 min. Fractions from each run containing product were pooled, acidified to pH 3 with glacial HOAc, concentrated in vacuo to a volume of ˜50 ml, and purified by preparative HPLC on a Waters C₁₈ Delta-Pak column 15 μM 300A (A=0.1% TFA/H₂O; B=0.1% TFA/CH₃CN), gradient elution 95·70% A/60 min, isocratic 70%/20 min. Homogeneous fractions (evaluated by HPLC, system A, 95·50% A) from all three runs were pooled and concentrated to a volume of ˜100 ml., diluted with 5% CH₃CN, and passed through AG4×4 ion exchange resin (acetate cycle), followed by freeze-drying to give the title compound as a lyophilized powder.

| | |
|---|---|
| HPLC conditions, | |
| system A: | |
| Column: | Vydac 15 cm #218TP5415, C18 |
| Eluant: | Gradient (A · B) over 45 min. |
| | A = 0.1% TFA/H₂O, B = 0.1% TFA/acetonitrile |
| Flow: | 1.5 ml/min. |

| | |
|---|---|
| Retention times: | |
| BDAM (dAc) vinbiastine | 23.5 min. |
| N-Acetyl-Ser—Ser—Ser—Chg—Gln—Ser—Val—OH | 14.5 min. |
| 4-Des-acetylvinblastine-23-( N-Acetyl-Ser—Ser— | 29.5 min. |
| Ser—Chg—Gln—Ser—Val—BDAM) amide | |
| High Resolution ES/FT-MS: | 1662.03 |

Amino Acid Compositional Analysis¹ (theory/found):

| | | | |
|---|---|---|---|
| ²Ser 4/3.6 | ³Glu 1/2.10 | ⁴Val 1/0.7 | Chg 1/0.95 |

| | |
|---|---|
| Peptide content | 0.504 μmol/mg |

Note:
¹20 hr, 100° C., 6N HCl
²Uncorrected
³Gln converted to Glu
⁴Incomplete hydrolysis

### EXAMPLE 8

Preparation of 4-des-Acetylvinblastine-23-(N-methoxy-diethylene-oxyacetyl-4-trans-L-Hyp-Ser-Ser-Chg-Gln-Ser-Val-BDAM) amide acetate salt

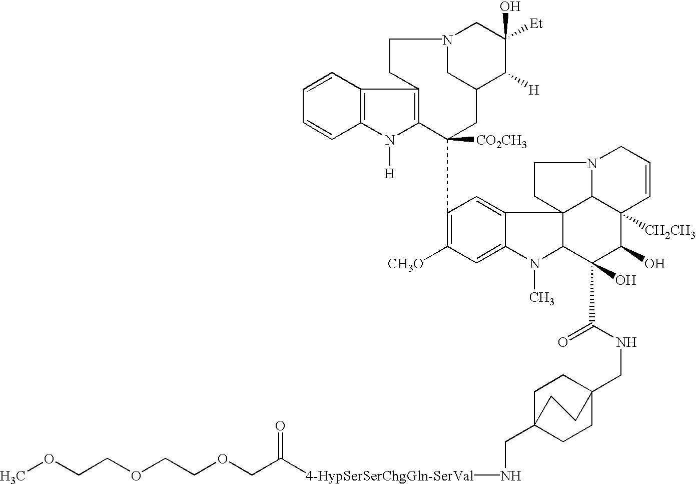

Step A:

N-methoxydiethyleneoxyacetyl-4-trans-L-Hyp-Ser-Ser-Chg-Gln-Ser-Val-PAM Resin

Starting with 0.5 mmole (0.68 g) of Boc-Val-PAM resin, the protected peptide was synthesized on a ABI model 430A peptide synthesizer. The protocol used a 4-fold excess (2.0 mmol) of each of the following protected amino acids: Boc-Ser(Bzl)-OH, Boc-Gln-OH, Boc-Chg-OH, Boc-4-trans-Hyp(Bzl)-OH; and 2-[2-(2-methoxyethoxy)-ethoxy]acetic acid (2 couplings). During each coupling cycle Boc protection was removed using TFA, followed by neutralization with DIEA. Coupling was achieved using DCC and HOBt activation in N-methyl-2-pyrrolidinone. At the completion of the synthesis, the peptide resin was dried to yield the title compound.

Step B:

N-methoxydiethyleneoxyacetyl-4-trans-L-Hyp-Ser-Ser-Chg-Gln-Ser-Val-OH

Two 0.5-mmol runs of the above peptide-resin (2.4 g) were combined and treated with liquid HF (40 ml) for 1.5 hours at 0° C. in the presence of anisole (4 ml). After evaporation of the HF, the residue was washed with ether, filtered and leached with 150 ml of H₂O in several portions, followed by preparative HPLC on a Waters C₁₈ Delta-Pak column 15 μM 100A (A=0.1% TFA/H₂O; B=0.1% TFA/CH₃CN), gradient elution 95·70% A/70 min, and pooling of homogeneous fractions and freeze drying to give the title compound as lyophilized powder. The sample purity was determined as 99% by HPLC.

Step C:

4-des-Acetylvinblastine-23-(N-methoxydiethylene-oxyacetyl-4-trans-L-Hvp-Ser-Ser-Chg-Gln-Ser-Val-BDAM) amide acetate salt

Samples of 440 mg (0.47 mmol) of the peptide from step B and 340 mg (0.33 mmol) of BDAM-(dAc)vinblastine, prepared as described in Example 6, Step F, were dissolved in 25 ml of DMF under N₂, cooling to 0° C. Then 85 mg (0.63 mmol) of 1-hydroxy-7-azabenzotriazole (HOAt) was added, and the pH was adjusted to 6.5-7 (moistened 5-10 range pH paper) with 2,4,6-collidine, followed by addition of 117 mg (0.61 mmol) of 1-(3-dimethylaminopropyl)-3-ethylcarbodiimide hydrochloride (EDC). Stirring was continued at 0-5° C. until completion of the coupling as monitored by analytical HPLC (A=0.1% TFA/H₂O; B=0.1% TFA/CH₃CN), maintaining the pH at 6.5-7 by periodic addition of 2,4,6-collidine. After 3 hours the reaction was worked up by addition of ˜10 ml of H₂O, stirring 1 hour and concentration to small volume in vacuo, then dissolution in ca. 70 ml of 5% HOAc. and preparative HPLC on a Waters C₁₈ Delta-Pak column 15 μM 300A (A=0.1% TFA/H₂O; B=0.1% TFA/CH₃CN), gradient elution 95·40% A/70 min). Homogeneous fractions (evaluated by HPLC, system A, 95·50% A) from all three runs were pooled and concentrated to a volume of ˜50 ml and passed through AG4×4 ion exchange resin (acetate cycle), followed by freeze-drying to give the title compound as a lyophilized powder.

| | |
|---|---|
| HPLC conditions, | |
| system A: | |
| Column: | Vydac 15 cm #218TP5415, C18 |
| Eluant: | Gradient (A · B) over 45 min. |
| | A = 0.1% TFA/H₂O, B = 0.1% TFA/acetonitrile |
| Flow: | 1.5 ml/min. |

| | |
|---|---|
| Retention times: | |
| BDAM (dAc) vinbiastine | 23.5 min. |
| N-methoxydiethyleneoxyacetyl-4-trans- | 16.2 min. |
| L-Hyp—Ser—Ser—Chg—Gln—Ser—Val—OH | |
| 4-des-Acetylvinblastine-23-(N-methoxydiethyleneoxyacetyl- | 29.6 min. |
| 4-trans-L-Hyp—Ser—Ser—Chg—Gln—Ser—Val—BDAM) | |
| amide | |
| High Resolution ES/FT-MS: | 1805.95 |

Amino Acid Compositional Analysis¹ (theory/found):

| | | | | |
|---|---|---|---|---|
| ²Ser 3/1.7 | ³Glu 1/1.01 | ⁴Val 1/0.93 | Chg 1/0.98 | Hyp 1/1.01 |

Peptide content = 0.497 μmol/mg
Note:
¹20 hr, 100° C., 6N HCl
²Uncorrected
³Gln converted to Glu
⁴Incomplete hydrolysis

### EXAMPLE 9

Preparation of 4-des-Acetylvinblastine-23-(N-Acetyl-4-trans-L-Hyp-Ser-Ser-Chg-Gln-Ser-HCAP) amide acetate salt (9-7)

Step A:

N-Acetyl-4-trans-L-Hyp-Ser-Ser-Chg-Gln-OH (9-1)

Starting with 0.5 mmole (0.80 g) of Fmoc-Gln(Trt)-Wang resin, the protected peptide was synthesized on a ABI model 430A peptide synthesizer. The protocol used a 4-fold excess (2.0 mmol) of each of the following protected amino acids: Fmoc-Ser(tBu)-OH, Fmoc-Chg-OH, Fmoc-4-trans-Hyp(tBu)-OH and acetic acid (2 couplings). During each coupling cycle Fmoc protection was removed using 20% piperidine in DMF. Coupling was achieved using DCC and HOBt activation in N-methyl-2-pyrrolidinone. At the completion of the synthesis, the peptide resin was dried. 1.3 g peptide-resin was treated with 95% TFA:2.5% H20:2.5%. Triisopropylsilane (20 ml) for 2 hours at room temperature under argon. After evaporation of the TFA, the residue was washed with ether, filtered and dried to give crude peptide which was purified by preparatory HPLC on a Delta-Pak Cl 8 column with 0.1% trifluoroacetic acid-aqueous acetonitrile solvent systems using 100-70%A, 60min linear gradient. Fractions containing product of at least 99% (HPLC) purity were combined to give the title compound.

FABMS: 615.3

Peptide Content: 1.03 mmole/mg.

HPLC: 99% pure @214 nm, retention time=10.16 min, (Vydac C₁₈, gradient of 95% A/B to 50% A/B over 30 min, A=0.1% TFA-H₂O, B=0.1% TFA-CH₃CN)

Step B:

N-Boc-(1S,2R)-(+)-Norephedrine (9-2)

A solution of 1.51 g (10 mmol) of (1S,2R)-(+)-Norephedrine in a mixture of 1,4 dioxane (20 ml), water (10 ml) and IN NaOH (10 ml) was stirred and cooled in an ice-water bath. Di-(t-butyl) dicarbonate (2.4 g, 11 mmol) was added in portions over approx. 20 minutes. The reaction was stirred in the cold for 2 hours, then at room temperature for an additional 1 hour. The solution was concentrated to remove most of the dioxane, cooled in an ice bath and covered with a layer of ethyl acetate (30 ml) and acidified to pH 2 with IN KHSO₄. The aqueous phase was extracted 2× with EtOAc. The combined extracts were washed with water, brine and were concentrated and dried to provide the desired product as a white crystalline solid (9-2). FABMS: 252

Step C:

N-Boc-HCAP (9-3)

A solution of 2.38 g of N-Boc-(lS,2R)-(+)-Norephedrine (9-2) in 50 ml acetic acid/10 ml H₂O was hydrogenated at 60 psi on a Parr apparatus over 500 mg of Ir black catalyst for 24 hours. The reaction was filtered through a Celite pad, and the filtrate concentrated in vacuo to give a tan foam (9-3). FABMS: 258.2

Step D:

N-Benzyloxycarbonyl-Ser-N-t-Boc-HCAP ester (9-4)

A solution of 1.95 g (6.6 mmol) of N-Z-Ser(tBu)-OH, 1.54g (6.0 mmol) of N-Boc-HCAP (9-3), 1.26 g (6.6 mmol) of EDC, and 146 mg (1.2 mmol) of DMAP in 30 ml of anh. CH₂Cl₂ was treated and the resulting solution stirred at room temperature in an N₂ atmosphere for 12 hours. The solvent was removed in vacuo, the residue dissolved in ethyl acetate (150 ml) and the solution extracted with 0.5 N NaHCO₃ (50 ml), water (50 ml) and brine, then dried and concentrated to provide the crude coupling product (9-4). Step E: H-Ser(tBu)-N-t-Boc-HCAP ester (9-5) A 2.0 g of (9-4) in a solution of 90 ml EtOH, 20 ml water, and 10 ml acetic acid was hydrogenated on a Parr apparatus at 50 psi over 200 mg of Pd(OH)₂ catalyst for 3 hours. The reaction was filtered through a Celite pad, and the filtrate was concentrated to small volume in vacuo, then purified by preparatory HPLC on a Delta-Pak C₁₈ column with 0.1% trifluoroacetic acid-aqueous acetonitrile solvent systems using 95-50%A, 60min linear gradient. Fractions containing product of at least 99% (HPLC) purity were combined to give the intermediate (9-5). FABMS: 401.3

Step F:

N-Acetyl-4-trans-L-Hyp-Ser-Ser-Chg-Gln-Ser-HCAP amine (9-6)

A solution of 614 mg (1.0 mmol) of N-Acetyl-4-trans-L Hyp-Ser-Ser-Chg-Gln-OH (9-1), 400 mg (1.0 mmol) of H-Ser(tBu)-N-t-Boc-HCAP ester (9-5), 229 mg (1.2 mmol) of EDC, and 81 mg (0.5 mmol) of ODBHT (3,4-dihydro-3-hydroxy-4-oxo-1,2,3-benzotriazine), in 7 ml of DMF was stirred at 0° C. in an N₂ atmosphere for 10 hours. The solvent was removed in vacuo, the residue was washed with ether and dried. The crude product was treated with 95% TFA:5% H₂O (20 ml) for 2 hours at room temperature under argon. After evaporation of the TFA, the residue was purified by preparatory HPLC on a Delta-Pak C₁₈ column with 0.1% trifluoroacetic acid-aqueous acetonitrile solvent systems using 95-50%A, 60min linear gradient. Fractions containing product of at least 99% (HPLC) purity were combined to give the intermediate compound (9-6).

FABMS: 841.8

Peptide Content: 863.39 NMole/mg.

HPLC: 99% pure @214 nm, retention time=13.7 min, (Vydac C₁₈, gradient of 95% A/B to 5% A/B over 30 min, A=0.1% TFA-H₂O, B=0.1% TFA-CH₃CN)

Step G:

4-des-Acetylvinblastine-23-(N-Ac-4-trans-L-Hyp-Ser-Ser-Chg-Gln-Ser-HCAP) amide acetate salt (9-7)

A solution of 0.461 of 4-des-acetylvinblastine-23-hydrazide (0.6 mmol) in 10 ml DMF cooled to −15° C. under Argon, was converted to the azide in situ by acidification with 4M HCl in dioxane to pH<1.5 (moistened 0-2.5 range paper), followed by addition of 0.105 ml (1.3 equiv) of isoamyl nitrite and stirring for 1 hour at 10-15° C. The pH was brought to 7 by the addition of DIEA, and 555 mg (0.66 mmol) of amine derivative (9-6) from step F was then added, and the reaction was stirred at 0° C. for 24 hours, and purified by preparatory HPLC on a 15 μM, 100 A, Delta-Pak C₁₈ column with 0.1% trifluoroacetic acid-aqueous acetonitrile solvent systems using 95-50%A, 60min linear gradient. Homogeneous fractions were pooled and concentrated in vacuo, followed by freeze-drying to give the title compound as the TFA salt which was converted to the corresponding HOAc salt by AG 4×4 resin (100-200 mesh, free base form, BIO-RAD) (18-7).

ES⁺: 1576.7

Peptide Content: 461.81 NMole/mg.

Ser 3.04;Hyp 1.07; Chg 1.02; Glu 1.00

HPLC: 99% pure @214 nm, retention time=18.31 min, (Vydac C₁₈, gradient of 95% A/B to 5% A/B over 30 min, A=0.1% TFA-H₂O, B-0.1% TFA-CH₃CN)

### EXAMPLE 10

Preparation of 4-des-Acetylvinblastine-23-(N-Acetyl-Ser-Chg-Gln-Ser-Ser-Pro-HCAP) amide acetate salt (10-5)

Step A:

N-Acetyl-Ser-Chz-Gln-Ser-Ser-OH (10-1)

Starting with 0.5 mmole (0.80 g) of Fmoc-Ser(tBu)-Wang resin, the protected peptide was synthesized on a ABI model 430A peptide synthesizer. The protocol used a 4-fold excess (2.0 mmol) of each of the following protected amino acids: Fmoc-Ser(tBu)-OH, Fmoc-Gln-OH, Fmoc-Chg-OH, Fmoc-Ser(tBu)-OH and acetic acid (2 couplings). During each coupling cycle Fmoc protection was removed using 20% piperidine in DMF. Coupling was achieved using DCC and HOBt activation in N-methyl-2-pyrrolidinone. At the completion of the synthesis, the peptide resin was dried. 1.3 g peptide-resin was treated with 95% TFA:2.5% H₂O:2.5% Triisopropylsilane (20 ml) for 2 hours at room temperature under argon. After evaporation of the TFA, the residue was washed with ether, filtered and dried to give crude peptide which was purified by preparatory HPLC on a Delta-Pak C₁₈ column with 0.1% trifluoroacetic acid-aqueous acetonitrile solvent systems using 100-70% A, 60min linear gradient. Fractions containing product of at least 99% (HPLC) purity were combined to give the title compound.

FABMS: 589.5

Peptide Content: 1.01 NMole/mg.

HPLC: 99% pure @214 nm, retention time=10.7 min, (Vydac C₁₈, gradient of 95% A/B to 50% A/B over 30 min, A=0.1% TFA-H₂O, B=0.1% TFA-CH₃CN)

Step B:

N-Boc-(1S,2R)-(+)-Norephedrine (9-2)

A solution of 1.51 g (10 mmol) of (1S,2R)-(+)-Norephedrine in a mixture of 1,4 dioxane (20 ml), water (10 ml) and IN NaOH (10 ml) is stirred and cooled in an ice-water bath. Di-(t-butyl) dicarbonate (2.4 g, 11 mmol) was added in portions over approx. 20 minutes. The reaction was stirred in the cold for 2 hours, then at room temperature for an additional 1 hour. The solution was concentrated to remove most of the dioxane, cooled in an ice bath and covered with a layer of ethyl acetate (30 ml) and acidified to pH 2 with IN KHSO₄. The aqueous phase was extracted 2× with EtOAc. The combined extracts were washed with water, brine and were concentrated and dried to provide the desired product as a white crystalline solid. FABMS: 252

Step C:

N-Boc-HCAP (9-3)

A solution of 2.38 g of N-Boc-(1S,2R)-(+)-Norephedrine (9-2) in 50 ml acetic acid/10 ml H₂O was hydrogenated at 60 psi on a Parr apparatus over 500 mg of Ir black catalyst for 24 hrs. The reaction was filtered through a Celite pad, and the filtrate concentrated in vacuo to give a tan foam. FABMS: 258.2

Step D:

N-Benzyloxycarbonyl-Pro-N-t-Boc-HCAP ester (10-2)

A solution of 1.62 g (6.6 mmol) of N-Z-Pro-OH, 1.54g (6.0 mmol) of N-Boc-HCAP (9-3), 1.26 g (6.6 mmol) of EDC, and 146 mg (1.2 mmol) of DMAP in 30 ml of anh. CH₂Cl₂ was treated and the resulting solution stirred at room temperature in an N₂ atmosphere for 12 hours. The solvent was removed in vacuo, the residue dissolved in ethyl acetate (150 ml) and the solution extracted with 0.5 N NaHCO₃ (50 ml), water (50 ml) and brine, then dried and concentrated to provide the crude coupling product.

Step E:

H-Pro-N-t-Boc-HCAP ester (10-3)

A 2.0 g of (10-2) in a solution of 90 ml EtOH, 20 ml water, and 10 ml acetic acid was hydrogenated on a Parr apparatus at 50 psi over 200 mg of Pd(OH)₂ catalyst for 3 hours. The reaction was filtered through a Celite pad, and the filtrate was concentrated to small volume in vacuo, then purified by preparatory HPLC on a Delta-Pak C₁₈ column with 0.1% trifluoroacetic acid-aqueous acetonitrile solvent systems using 95-50% A, 60min linear gradient. Fractions containing product of at least 99% (HPLC) purity were combined to give the title compound (10-3).

FABMS: 356.3

Step F:

N-Acetyl-Ser-Chg-Gln-Ser-Ser-Pro-HCAP amine (10-4)

A solution of 589 mg (1.0 mmol) of N-Acetyl-Ser-Chg-Gln-Ser-Ser-OH (10-1), 356 mg (1.0 mmol) of H-Pro-N-t-Boc-HCAP ester (10-3), 229 mg (1.2 mmol) of EDC, and 81 mg (0.5 mmol) of ODBHT (3,4-dihydro-3-hydroxy-4-oxo-1,2,3-benzotriazine), in 7 ml of DMF was stirred at 0° C. in an N₂ atmosphere for 10 hours. The solvent was removed in vacuo, the residue was washed with ether and dried. The crude product was treated with 95% TFA:5% H₂O (20 ml) for 2 hours at room temperature under argon. After evaporation of the TFA, the residue was purified by preparatory HPLC on a Delta-Pak C₁₈ column with 0.1% trifluoroacetic acid-aqueous acetonitrile solvent systems using 95-50% A, 60min linear gradient. Fractions containing product of at least 99% (HPLC) purity were combined to give the title compound (10-4).

FABMS: 825.5

Peptide Content: 893.6 NMole/mg.

HPLC: 99% pure @214 nm, retention time=15.2 min, (Vydac C₁₈, gradient of 95% A/B to 5% A/B over 30 min, A=0.1% TFA-H₂O, B=0.1% TFA-CH₃CN)

Step G:

4-des-Acetylvinblastine-23-(N-Ac-Ser-Chg-Gln-Ser-Ser-Pro-HCAP) amide acetate salt (10-5)

A solution of 0.461 of 4-des-acetylvinblastine-23-hydrazide (0.6 mmol) in 10 ml DMF cooled to −15° C. under Argon, was converted to the azide in situ by acidification with 4M HCl in dioxane to pH<1.5 (moistened 0-2.5 range paper), followed by addition of 0.105 ml (1.3 equiv) of isoamyl nitrite and stirring for 1 hour at 10-15° C. The pH was brought to 7 by the addition of DIEA, and 545 mg (0.66 mmol) of amine derivative (10-4) from step F was then added, and the reaction was stirred at 0° C. for 24 hours, and purified by preparatory HPLC on a 15 μM, 100 A, Delta-Pak C₁₈ column with 0.1% trifluoroacetic acid-aqueous acetonitrile solvent systems using 95-50%A, 60min linear gradient. Homogeneous fractions were pooled and concentrated in vacuo, followed by freeze-drying to give the title compound as the TFA salt which was converted to title compound by AG 4×4 resin (100-200 mesh, free base form, BIO-RAD) (19-5)

ES⁺: 1560.9

Peptide Content: 586.8 NMole/mg.

Ser 3.04; Chg 1.01; Glu 1.00; Pro 0.97

HPLC: 99% pure @214 nm, retention time=13.4 min, (Vydac C₁₈, gradient of 95% A/B to 5% A/B over 30 min, A=0.1% TFA-H₂O, B=0.1% TFA-CH₃CN)

### PSA CONJUGATE ASSAYS

### EXAMPLE 11

Assessment of the Recognition of Oligopeptide-Cytotoxic Drug Conjugates by Free PSA

The PSA conjugates, prepared as described above and in particular in Examples 1-10, are individually dissolved in PSA digestion buffer (50 mM tris(hydroxymethyl)-aminomethane pH7.4, 140 mM NaCl) and the solution added to PSA at a molar ration of 100 to 1. Alternatively, the PSA digestion buffer utilized is 50 mM tris(hydroxymethyl)-aminomethane pH7.4, 140 mM NaCl. The reaction is quenched after various reaction times by the addition of trifluoroacetic acid (TFA) to a final 1% (volume/volume). Alternatively the reaction is quenched with 10 mM ZnCl₂. The quenched reaction is analyzed by HPLC on a reversed-phase C₁₈ column using an aqueous 0.1% TFA/acetonitrile gradient. The amount of time (in minutes) required for 50% cleavage of the noted oligopeptide-cytotoxic agent conjugates with enzymatically active free PSA were then calculated.

### EXAMPLE 12

In vitro Assay of Cytotoxicity of Peptidyl Derivatives of Doxorubicin

The cytotoxicities of a cleaveable oligopeptide-doxorubicin conjugates, prepared as described above and in particular in Examples 1-3, against a line of cells which is known to be killed by unmodified doxorubicin are assessed with an Alamar Blue assay. Specifically, cell cultures of LNCap prostate tumor cells (which express enzymatically active PSA) or DuPRO cells in 96 well plates are diluted with medium (Dulbecco's Minimum Essential Medium-α[MEM-α]) containing various concentrations of a given conjugate (final plate well volume of 200 μl). The cells are incubated for 3 days at 37° C., 20 μl of Alamar Blue is added to the assay well. The cells are further incubated and the assay plates are read on a EL-310 ELISA reader at the dual wavelengths of 570 and 600 nm at 4 and 7 hours after addition of Alamar Blue. Relative percentage viability at the various concentration of conjugate tested is then calculated versus control (no conjugate) cultures.

### EXAMPLE 13

In vitro Assay of Cytotoxicity of Peptidyl Derivatives of Vinca Drugs

The cytotoxicities of a cleaveable oligopeptide-vinca drug conjugates, prepared as described above and in particular in Examples 4-10, against a line of cells which is known to be killed by unmodified vinca drug was assessed with an Alamar Blue assay. Specifically, cell cultures of LNCap prostate tumor cells, Colo320DM cells (designated C₃₂₀) or T47D cells in 96 well plates are diluted with medium containing various concentrations of a given conjugate (final plate well volume of 200μl). The Colo320DM cells, which do not express free PSA, are used as a control cell line to determine non-mechanism based toxicity. The cells are incubated for 3 days at 37° C., 20 μl of Alamar Blue is added to the assay well. The cells are further incubated and the assay plates are read on a EL-310 ELISA reader at the dual wavelengths of 570 and 600 nm at 4 and 7 hours after addition of Alamar Blue. Relative percentage viability at the various concentration of conjugate tested is then calculated versus control (no conjugate) cultures and an EC₅₀ was determined.

### EXAMPLE 14

In vivo Efficacy of Peptidyl-Cytotoxic Agent Conjugates

LNCAP.FGC or DuPRO-1 cells are trypsinized, resuspended in the growth medium and centifuged for 6 mins. at 200× g. The cells are resuspended in serum-free α-MEM and counted. The appropriate volume of this solution containing the desired number of cells is then transferred to a conical centrifuge tube, centrifuged as before and resuspended in the appropriate volume of a cold 1:1 mixture of α-MEM-Matrigel. The suspension is kept on ice until the animals are inoculated.

Harlan Sprague Dawley male nude mice (10-12 weeks old) are restrained without anesthesia and are inoculated with 0.5 ML of cell suspension on the left flank by subcutaneous injection using a 22G needle. Mice are either given approximately 5×10⁵ DuPRO cells or 1.5×10⁷ LNCaP.FGC cells.

Following inoculation with the tumor cells the mice are treated under one of two protocols:

Protocol A:

One day after cell inoculation the animals are dosed with a 0.1-0.5 mL volume of test conjugate, unconjugated cytotoxic agent or vehicle control (sterile water). Dosages of the conjugate and unconjugated cytotoxic agent are initially the maximum non-lethal amount, but may be subsequently titrated lower. Identical doses are administered at 24 hour intervals for 5 days. After 10 days, blood samples are removed from the mice and the serum level of PSA is determined. Similar serum PSA levels are determined at 5-10 day intervals. At the end of 5.5 weeks the mice are sacrificed and weights of any tumors present are measured and serum PSA again determined. The animals' weights are determined at the beginning and end of the assay.

Protocol B:

Ten days after cell inoculation, blood samples are removed from the animals and serum levels of PSA are determined. Animals are then grouped according to their PSA serum levels. At 14-15 days after cell inoculation, the animals are dosed with a 0.1-0.5 mL volume of test conjugate, unconjugated cytotoxic agent or vehicle control (sterile water). Dosages of the conjugate and unconjugated cytotoxic agent are initially the maximum non-lethal amount, but may be subsequently titrated lower. Identical doses are administered at 24 hour intervals for 5 days. Serum PSA levels are determined at 5-10 day intervals. At the end of 5.5 weeks the mice are sacrificed, weights of any tumors present are measured and serum PSA again determined. The animals' weights are determined at the beginning and end of the assay.

### EXAMPLE 15

In vitro determination of proteolytic cleavage of conjugates by endogenous non-PSA proteases

Step A:

Preparation of proteolytic tissue extracts

All procedures are carried out at 4° C. Appropriate animals are sacrificed and the relevant tissues are isolated and stored in liquid nitrogen. The frozen tissue is pulverized using a mortar and pestle and the pulverized tissue is transfered to a Potter-Elvejeh homogenizer and 2 volumes of Buffer A (50 mM Tris containing 1.15% KCl, pH 7.5) are added. The tissue is then disrupted with 20 strokes using first a lose fitting and then a tight fitting pestle. The homogenate is centrifuged at 10,000×g in a swinging bucket rotor (HB4-5), the pellet is discarded and the re-supematant centrifuged at 100,000×g (Ti 70). The supernatant (cytosol) is saved.

The pellet is resuspended in Buffer B (10 mM EDTA containing 1.15% KCl, pH 7.5) using the same volume used in step as used above with Buffer A. The suspension is homogenized in a dounce homogenizer and the solution centrifuged at 100,000× g. The supernatant is discarded and the pellet resuspended in Buffer C (10 mM potassium phosphate buffer containing 0.25 M sucrose, pH 7.4), using ½ the volume used above, and homogenized with a dounce homogenizer.

Protein content of the two solutions (cytosol and membrane) is determined using the Bradford assay. Assay aliquots are then removed and frozen in liquid N₂. The aliquots are stored at −70° C.

Step B:

Proteolytic cleavage assay

For each time point, 20 microgram of PSA conjugate and 150 micrograms of tissue protein, prepared as described in Step A and as determined by Bradford in reaction buffer are placed in solution of final volume of 200 microliters in buffer (50 mM TRIS, 140 mM NaCl, pH 7.2). Assay reactions are run for 0, 30, 60, 120, and 180 minutes and are then quenched with 9 microliters of 0.1 M ZnCl₂ and immediately placed in boiling water for 90 seconds. Reaction products are analyzed by HPLC using a VYDAC C₁₈ 15 cm column in water/acetonitrile (5% to 50% acetonitrile over 30 minutes).

### EXAMPLE 16

In Vivo Efficacy of Administration of a PSA Conjugate and Radiation Therapy

Male nude mice (4 groups of 15) are injected subcutaneously with 1.5×10 LNCaP.FGC cells (available from the American Type Culture Collection, ATCC No. CRL-1740; see also J. S. Horoszewicz et al. Cancer Res., 43:1809-1818 (1983)) in 80% Matrigel.

Five days after the tumor cell implantation, a solution of test PSA conjugate is administered. The PSA conjugate is administered IV as a therapeutically minimal dose. For example, when the PSA conjugate described in Example 2 is tested, a 0.20 mL of a solution of test PSA conjugate, (3-5 mpk, 34.1 mL D5W+80 μL 7.5% sodium bicarbonate) is administered. After administration of the PSA conjugate, a therapeutically effective amount of radiation therapy is delivered.

After the initial dose of PSA conjugate, the animals may be administered PSA conjugate solution either as four additional doses (one/day) over four consecutive days, or once a week for four consecutive weeks. Additionally, the dosage may be increased up to the maximum tolerated dose (MTD). For example, if Compound B of Example 2 is tested, up to 40 mpk may be administered.

At the end of 5-6 weeks after the inoculation with the LNCaP cells the mice are bled from the tail vein and the plasma PSA level is measured using a Tandem®-E PSA ImmunoEnzyMetri Assay kit (Hybritech). The mice are then sacrificed, weighed, tumors excised and weighed.

What is claimed is:

1. A method for treating cancer in a mammal in need thereof which comprises administering to said mammal amounts of at least one PSA conjugate in combination with radiation therapy.

2. The method according to claim 1 wherein an amount of an PSA conjugate is administered sequentially with radiation therapy.

3. The method according to claim 1 wherein an amount of an PSA conjugate is administered concomitantly with radiation therapy.

4. The method according to claim 1 wherein the cancer is a cancer related to cells that express enzymatically active PSA.

5. The method according to claim 1 wherein the cancer is prostate cancer.

6. The method according to claim 1 wherein the PSA conjugate is selected from:
a) a compound represented by the formula I: 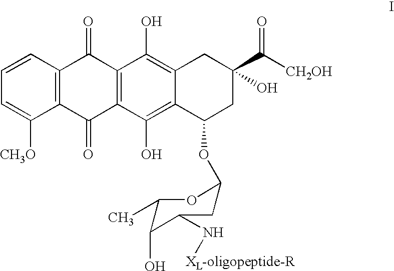
wherein:
oligopeptide is an oligopeptide which is specifically recognized by the free prostate specific antigen (PSA) and is capable of being proteolytically cleaved by the enzymatic activity of the free prostate specific antigen;
X_{L} is absent or is an amino acid selected from:
a) phenylalanine,
b) leucine,
c) valine,
d) isoleucine,
e) (2-naphthyl)alanine,
f) cyclohexylalanine,
g) diphenylalanine,
h) norvaline, and
j) norleucine;
R is hydrogen or —(C═O)R¹; and
R¹ is C₁-C₆-alkyl or aryl,
or the pharmaceutically acceptable salt thereof;
b) a compound represented by the formula II: 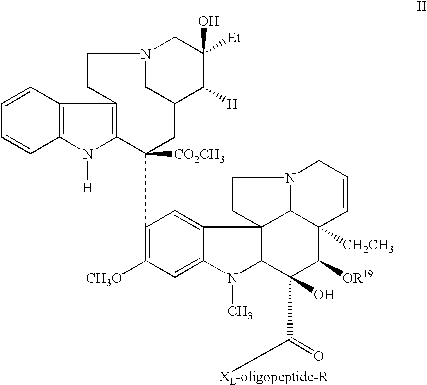
wherein:
oligopeptide is an oligopeptide which is specifically recognized by the free prostate specific antigen (PS A) and is capable of being proteolytic ally cleaved by the enzymatic activity of the free prostate specific antigen;
X_{L} is absent or is an amino acid selected from:
a) phenylalanine,
b) leucine,
c) valine,
d) isoleucine,
e) (2-naphthyl)alanine,
f) cyclohexylalanine,
g) diphenylalanine,
h) norvaline, and
j) norleucine; or
X_{L} is —NH—(CH₂)ₙ—NH—;
R is hydrogen or —(C═O)R^{1;}
R¹ is C₁-C₆-alkyl or aryl;
R¹⁹ is hydrogen or acetyl; and
n is 1,2,3,4 or 5,
or the pharmaceutically acceptable salt thereof;
c) a compound represented by the formula III: 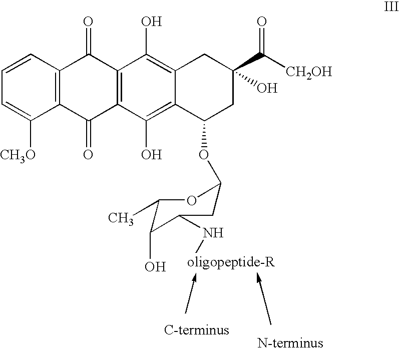
wherein:
oligopeptide is an oligopeptide which is selectively recognized by the free prostate specific antigen (PSA) and is capable of being proteolytically cleaved by the enzymatic activity of the free prostate specific antigen, wherein the oligopeptide comprises a cyclic amino acid of the formula: 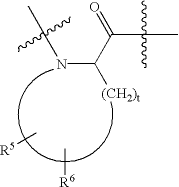
and wherein
the C-terminus carbonyl is covalently bound to the amine of doxorubicin;
R is selected from
a) hydrogen,
b) —(C═O)R^{1a}, 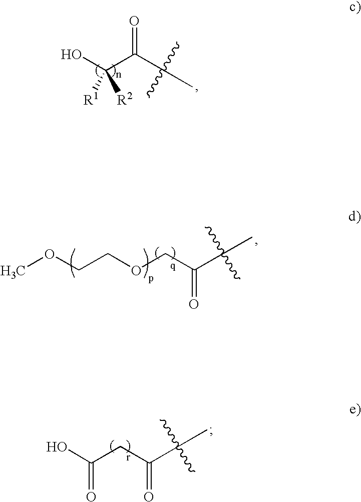
R¹ and R²are independently selected from: hydrogen, OH, C₁-C₆ alkyl, C₁-C₆ alkoxy, C₁-C₆ aralkyl and aryl;
R^{1a} is C₁-C₆-alkyl, hydroxylated aryl, polyhydroxylated aryl or aryl;
R⁵ is selected from HO— and C₁-C₆ alkoxy;
R⁶ is selected from hydrogen, halogen, C₁-C₆ alkyl, HO— and C₁-C₆ alkoxy; and
n is 1,2,3 or 4;
p is zero or an integer between 1 and 100;
q is 0 or 1, provided that if p is zero, q is 1;
r is an integer between 1 and 10; and
t is 3 or 4;
or a pharmaceutically acceptable salt thereof;
d) a compound represented by the formula IV: 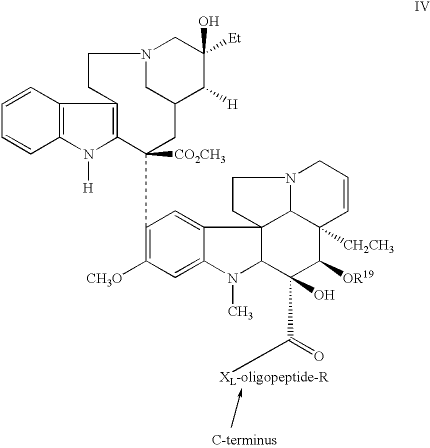
wherein:
oligopeptide is an oligopeptide which is specifically recognized by the free prostate specific antigen (PSA) and is capable of being proteolytically cleaved by the enzymatic activity of the free prostate specific antigen, and the oligopeptide comprises a cyclic amino acid of the formula: 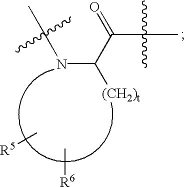
X_{L} is —NH—(CH₂)ᵤ—NH—;
R is selected from
a) hydrogen,
b) —(C═O)R^{1a}, 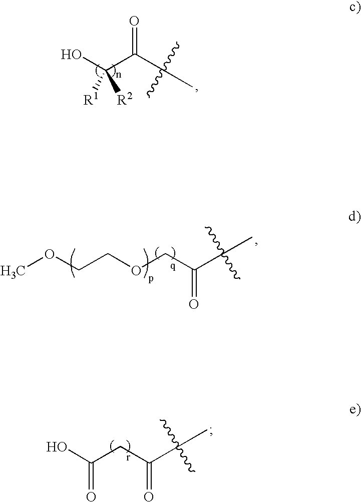
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated aryl, polyhydroxylated aryl or aryl;
R¹⁹ is hydrogen, (C₁-C₃ alkyl)—CO, or chlorosubstituted (C₁-C₃ alkyl)—CO;
n is 1,2,3 or 4;
p is zero or an integer between 1 and 100;
q is 0 or 1, provided that if p is zero, q is 1;
r is 1, 2 or 3;
t is 3 or 4;
u is 1, 2, 3, 4 or 5;
or the pharmaceutically acceptable salt thereof;
e) a compound represented by the formula V: 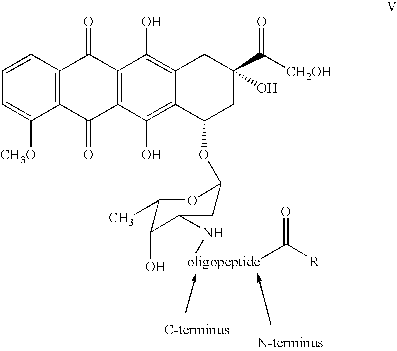
wherein:
oligopeptide is an oligopeptide which is selectively recognized by the free prostate specific antigen (PSA) and is capable of being proteolytically cleaved by the enzymatic activity of the free prostate specific antigen, and wherein the C-terminus carbonyl is covalently bound to the amine of doxorubicin and the N-terminus amine is covalently bound to the carbonyl of the blocking group;
R is selected from 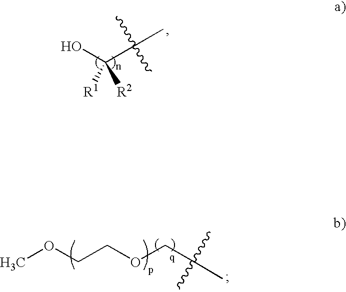
R¹ and R² are independently selected from: hydrogen, OH, C₁-C₆ alkyl, C₁-C₆ alkoxy, C₁-C₆ aralkyl and aryl;
n is 1,2,3 or 4;
p is zero or an integer between 1 and 100;
q is 0 or 1, provided that if p is zero, q is 1;
or the pharmaceutically acceptable salt thereof;
f) a compound represented by the formula VI: 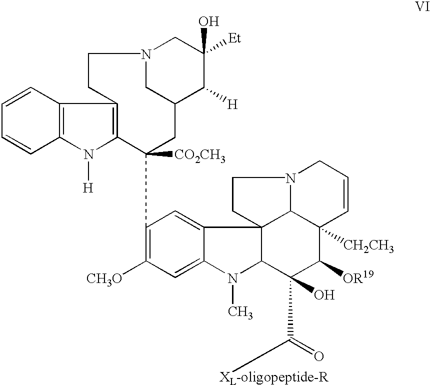
wherein:
oligopeptide is an oligopeptide which is specifically recognized by the free prostate specific antigen (PSA) and is capable of being proteolytically cleaved by the enzymatic activity of the free prostate specific antigen;
X_{L} is —NH—(CH₂)ᵣ—NH—;
R is selected from 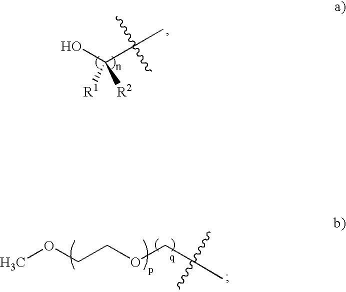
R¹ and R² are independently selected from: hydrogen, OH, C₁-C₆ alkyl, C₁-C₆ alkoxy, C₁-C₆ aralkyl and aryl;
R¹⁹ is hydrogen, (C₁-C₃ alkyl)—CO, or chlorosubstituted (C₁-C₃ alkyl)—CO;
n is 1,2,3 or 4;
p is zero or an integer between 1 and 100;
q is 0 or 1, provided that if p is zero, q is 1;
r is 1,2,3,4 or 5;
or the pharmaceutically acceptable salt thereof;
g) a compound represented by the formula VII: 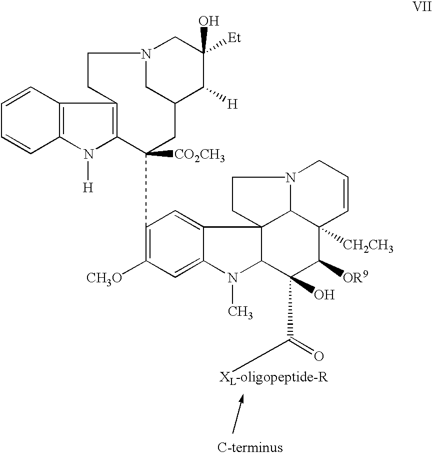
wherein:
oligopeptide is an oligopeptide which is specifically recognized by the free prostate specific antigen (PSA) and is capable of being proteolytically cleaved by the enzymatic activity of the free prostate specific antigen,
X_{L} is —NH—(CH₂)ᵤ—W—(CH₂)ᵤ—NH—;
R is selected from
a) hydrogen,
b) —(C═O)R^{1a}, 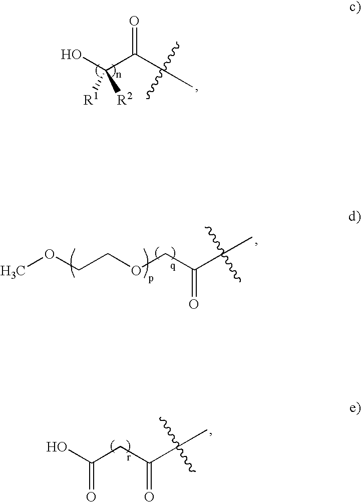
f) ethoxysquarate, and
g) cotininyl;
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;
R⁹ is hydrogen, (C₁-C₃ alkyl)—CO, or chlorosubstituted (C₁-C₃ alkyl)—CO;
W is selected from cyclopentyl, cyclohexyl, cycloheptyl or bicyclo[2.2.2]octanyl;
n is 1,2,3 or 4;
p is zero or an integer between 1 and 100;
q is 0 or 1, provided that if p is zero, q is 1;
r is 1,2 or 3;
t is 3 or 4;
u is 0, 1, 2 or 3;
or the pharmaceutically acceptable salt thereof; and
h) a compound represented by the formula VIII: 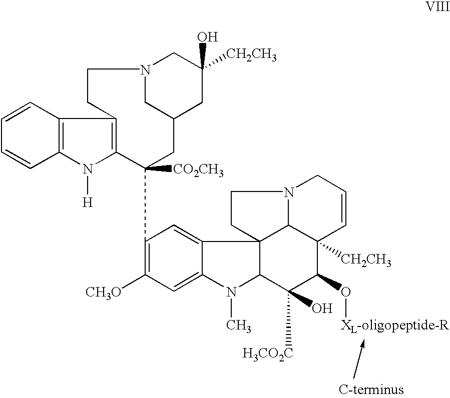
wherein:
oligopeptide is an oligopeptide which is specifically recognized by the free prostate specific antigen (PSA) and is capable of being proteolytically cleaved by the enzymatic activity of the free prostate specific antigen,
X_{L} is selected from: a bond, —C(O)—(CH₂)ᵤ—W—(CH₂)ᵤ—O— and —C(O)—(CH₂)ᵤ—W—(CH₂)ᵤ—NH—;
R is selected from
a) hydrogen,
b) —(C═O)R^{1a}, 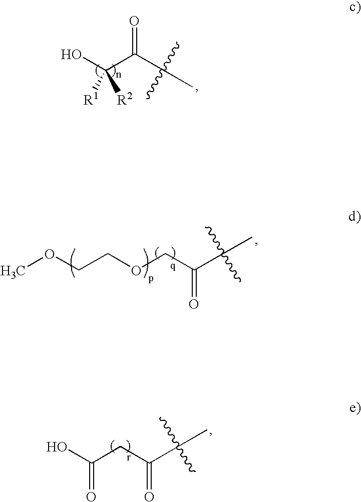
f) ethoxysquarate, and
g) cotininyl;
R¹ and R² are independently selected from: hydrogen, OH, C₁-C₆ alkyl, C₁-C₆ alkoxy, C₁-C₆ aralkyl and aryl;
R^{1a} is C₁-C₆-alkyl, hydroxylated C₃-C₈-cycloalkyl, polyhydroxylated C₃-C₈-cycloalkyl, hydroxylated aryl, polyhydroxylated aryl or aryl;
R⁹ is hydrogen, (C₁-C₃ alkyl)—CO, or chlorosubstituted (C₁-C₃ alkyl)—CO;
W is selected from a branched or straight chain C₁-C₆-alkyl, cyclopentyl, cyclohexyl, cycloheptyl or bicyclo[2.2.2]octanyl;
n is 1, 2,3 or 4;
p is zero or an integer between 1 and 100;
q is 0 or 1, provided that if p is zero, q is 1;
r is 1,2 or 3;
t is 3 or 4;
u is 0, 1,2 or 3;
or the pharmaceutically acceptable salt or optical isomer thereof.

7. The method according to claim 6 wherein the PSA conjugate is selected from: 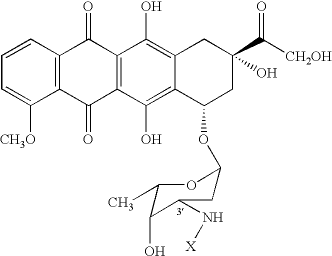
wherein X is:

| | |
|---|---|
| AsnLysIleSerTyrGlnSer- | (SEQ.ID.NO.:1), |
| AsnLysIleSerTyrGlnSerSer- | (SEQ.ID.NO.:2), |
| AsnLysIleSerTyrGlnSerSerSer- | (SEQ.ID.NO.:3), |
| AsnLysIleSerTyrGlnSerSerSerThr- | (SEQ.ID.NO.:4), |
| AsnLysIleSerTyrGlnSerSerSerThrGlu- | (SEQ.ID.NO.:5), |
| AlaAsnLysLleSerTyrGlnSerSerSerThrGlu- | (SEQ.ID.NO.:6), |
| Ac-AlaAsnLysIleSerTyrGlnSerSerSerThr- | (SEQ.ID.NO.:7), |
| Ac-AlaAsnLysIleSerTyrGlnSerSerSerThrLeu- | (SEQ.ID.NO.:8), |
| Ac-AlaAsnLysAlaSerTyrGlnSerAlaSerThrLeu- | (SEQ.ID.NO.:9), |
| Ac-AlaAsnLysAlaSerTyrGlnSerAlaSerLeu- | (SEQ.ID.NO.:10), |
| Ac-AlaAsnLysAlaSerTyrGlnSerSerSerLeu- | (SEQ.ID.NO.:11), |
| Ac-AlaAsnLysAlaSerTyrGlnSerSerLeu- | (SEQ.ID.NO.:12), |
| Ac-SerTyrGlnSerSerSerLeu- | (SEQ.ID.NO.:13), |
| Ac-hArgTyrGlnSerSerSerLeu- | (SEQ.ID.NO.:14). |
| Ac-LysTyrGlnSerSerSerLeu- | (SEQ.ID.NO.:15), or |
| Ac-LysTyrGlnSerSerNle- | (SEQ.ID.NO.:16); |

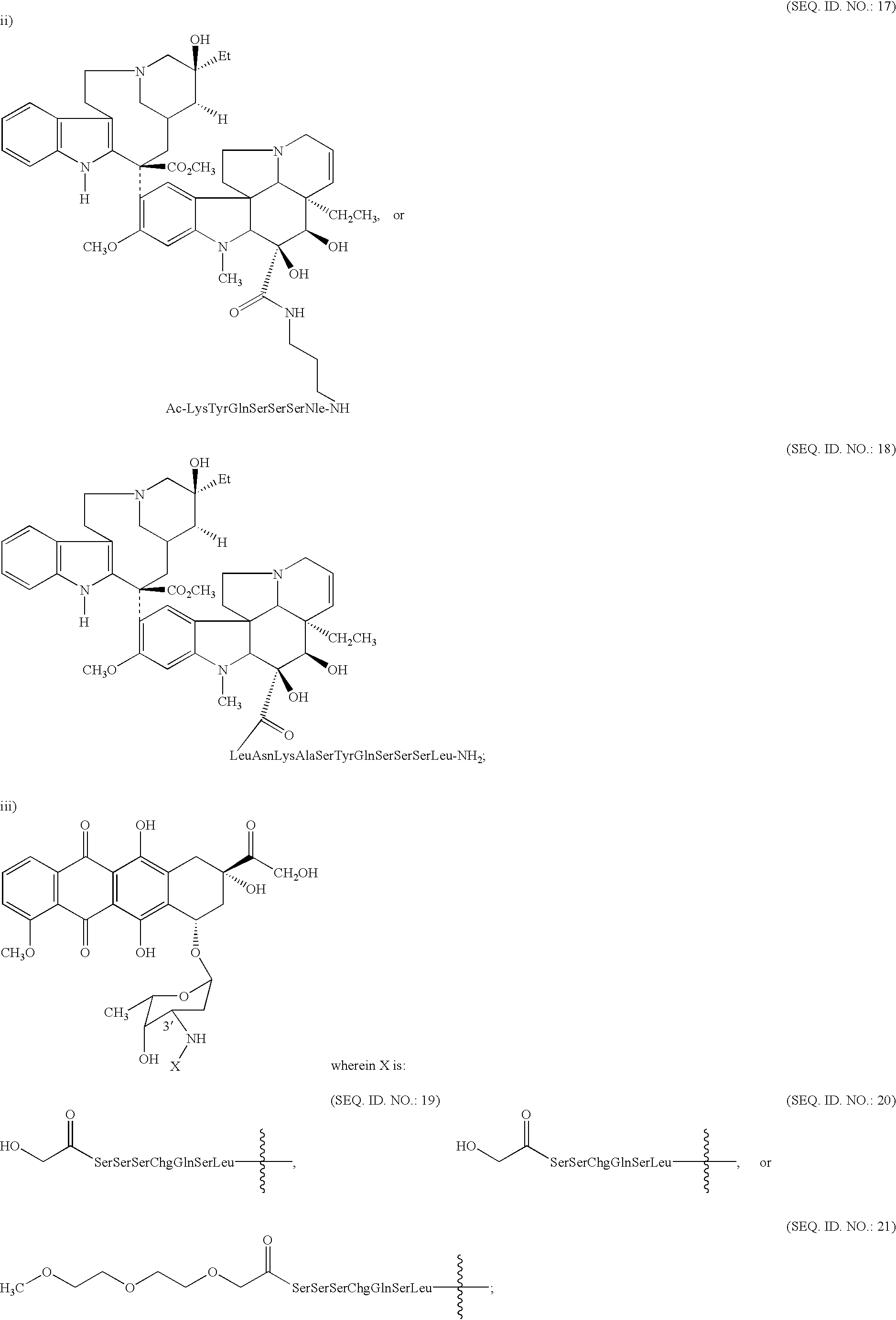

8. A method for treating prostatic disease in a mammal in need thereof which comprises administering to said mammal amounts of at least one PSA conjugate in combination with radiation therapy.

9. The method according to claim 8 herein the prostatic disease is selected from benign prostatic hyperplasia, prostatic intraepithelial meoplasia and prostate cancer.


SEQUENCE LISTING (46 sequences)

SEQ ID NO: 1 - protein, 7 aa, Artificial Sequence
  completely synthetic amino acid sequence
NKISYQS

SEQ ID NO: 2 - protein, 8 aa, Artificial Sequence
  completely synthetic amino acid sequence
NKISYQSS

SEQ ID NO: 3 - protein, 9 aa, Artificial Sequence
  completely synthetic amino acid sequence
NKISYQSSS

SEQ ID NO: 4 - protein, 10 aa, Artificial Sequence
  completely synthetic amino acid sequence
NKISYQSSST

SEQ ID NO: 5 - protein, 11 aa, Artificial Sequence
  completely synthetic amino acid sequence
NKISYQSSSTE

SEQ ID NO: 6 - protein, 12 aa, Artificial Sequence
  completely synthetic amino acid sequence
ANKISYQSSSTE

SEQ ID NO: 7 - protein, 11 aa, Artificial Sequence
  completely synthetic amino acid sequence
ANKISYQSSST

SEQ ID NO: 8 - protein, 12 aa, Artificial Sequence
  completely synthetic amino acid sequence
ANKISYQSSSTL

SEQ ID NO: 9 - protein, 12 aa, Artificial Sequence
  completely synthetic amino acid sequence
ANKASYQSASTL

SEQ ID NO: 10 - protein, 11 aa, Artificial Sequence
  completely synthetic amino acid sequence
ANKASYQSASL

SEQ ID NO: 11 - protein, 11 aa, Artificial Sequence
  completely synthetic amino acid sequence
ANKASYQSSSL

SEQ ID NO: 12 - protein, 10 aa, Artificial Sequence
  completely synthetic amino acid sequence
ANKASYQSSL

SEQ ID NO: 13 - protein, 7 aa, Artificial Sequence
  completely synthetic amino acid sequence
SYQSSSL

SEQ ID NO: 14 - protein, 7 aa, Artificial Sequence
  completely synthetic amino acid sequence
RYQSSSL

SEQ ID NO: 15 - protein, 7 aa, Artificial Sequence
  completely synthetic amino acid sequence
KYQSSSL

SEQ ID NO: 16 - protein, 6 aa, Artificial Sequence
  completely synthetic amino acid sequence
KYQSSL

SEQ ID NO: 17 - protein, 7 aa, Artificial Sequence
  completely synthetic amino acid sequence
KYQSSSL

SEQ ID NO: 18 - protein, 11 aa, Artificial Sequence
  completely synthetic amino acid sequence
LNKASYQSSSL

SEQ ID NO: 19 - protein, 7 aa, Artificial Sequence
  completely synthetic amino acid sequence
XSSXQSL

SEQ ID NO: 20 - protein, 6 aa, Artificial Sequence
  completely synthetic amino acid sequence
XSXQSL

SEQ ID NO: 21 - protein, 7 aa, Artificial Sequence
  completely synthetic amino acid sequence
XSSXQSL

SEQ ID NO: 22 - protein, 7 aa, Artificial Sequence
  completely synthetic amino acid sequence
XASXQSL

SEQ ID NO: 23 - protein, 7 aa, Artificial Sequence
  completely synthetic amino acid sequence
XASXQSL

SEQ ID NO: 24 - protein, 7 aa, Artificial Sequence
  completely synthetic amino acid sequence
PASXQSL

SEQ ID NO: 25 - protein, 7 aa, Artificial Sequence
  completely synthetic amino acid sequence
XASXQSL

SEQ ID NO: 26 - protein, 7 aa, Artificial Sequence
  completely synthetic amino acid sequence
XASXQSL

SEQ ID NO: 27 - protein, 7 aa, Artificial Sequence
  completely synthetic amino acid sequence
XASXQSL

SEQ ID NO: 28 - protein, 7 aa, Artificial Sequence
  completely synthetic amino acid sequence
XASXQSX

SEQ ID NO: 29 - protein, 7 aa, Artificial Sequence
  completely synthetic amino acid sequence
XASXQSL

SEQ ID NO: 30 - protein, 7 aa, Artificial Sequence
  completely synthetic amino acid sequence
XASXQSV

SEQ ID NO: 31 - protein, 7 aa, Artificial Sequence
  completely synthetic amino acid sequence
PASXQSL

SEQ ID NO: 32 - protein, 7 aa, Artificial Sequence
  completely synthetic amino acid sequence
SSSXQSV

SEQ ID NO: 33 - protein, 7 aa, Artificial Sequence
  completely synthetic amino acid sequence
XSSXQSV

SEQ ID NO: 34 - protein, 7 aa, Artificial Sequence
  completely synthetic amino acid sequence
SSSXQSL

SEQ ID NO: 35 - protein, 7 aa, Artificial Sequence
  completely synthetic amino acid sequence
XSSXQSL

SEQ ID NO: 36 - protein, 8 aa, Artificial Sequence
  completely synthetic amino acid sequence
XSSXQSSP

SEQ ID NO: 37 - protein, 7 aa, Artificial Sequence
  completely synthetic amino acid sequence
XSSXQSG

SEQ ID NO: 38 - protein, 8 aa, Artificial Sequence
  completely synthetic amino acid sequence
XSSXQSSG

SEQ ID NO: 39 - protein, 8 aa, Artificial Sequence
  completely synthetic amino acid sequence
XSSXQSSP

SEQ ID NO: 40 - protein, 7 aa, Artificial Sequence
  completely synthetic amino acid sequence
XSSXQSV

SEQ ID NO: 41 - protein, 8 aa, Artificial Sequence
  completely synthetic amino acid sequence
XSSXQSSP

SEQ ID NO: 42 - protein, 7 aa, Artificial Sequence
  completely synthetic amino acid sequence
XSSXQSP

SEQ ID NO: 43 - protein, 7 aa, Artificial Sequence
  completely synthetic amino acid sequence
XSSXQSP

SEQ ID NO: 44 - protein, 8 aa, Artificial Sequence
  completely synthetic amino acid sequence
XSSXQSSP

SEQ ID NO: 45 - protein, 7 aa, Artificial Sequence
  completely synthetic amino acid sequence
XSSXQSV

SEQ ID NO: 46 - protein, 7 aa, Artificial Sequence
  completely synthetic amino acid sequence
XSSXQSL